SEQUENCE LISTING (13 sequences)

SEQ ID NO: 1 - protein, 57 aa, Homo sapiens
EVCSEQAETGPCRAMISRWYFDVTEGKCAPFFYGGCGGNRNNGDTEEYCMAVCGSAI

SEQ ID NO: 2 - protein, 58 aa, Homo sapiens [sequence omitted]

SEQ ID NO: 3 - DNA, 853 bp, Homo sapiens
gggcctggagctgcacccgcttctgggtggacgcacttggcgagcggcgcgggatgcagacggctgcgaggcgctgggcacagttgctgtcccctttgacgatgatgacaagattgttgggggctacacctgtgaggagaattctctcccctaccaggtgtccctgaattctggctcccacttctgcggtggctccctcatcagcgaacagtgggtggtatcagcagctcactgctacaagacccgcatccaggtgagactgggagagcacaacatcaaagtcctggaggggaatgagcagttcatcaatgcggccaagatcatccgccaccctaaatacaacagggacactctggacaatgacatcatgctgatcaaactctcctcacctgccgtcatcaatgcccgcgtgtccaccatctctctgcccaccgcccctccagctgctggcactgagtgcctcatctccggctggggcaacactctgagctttggtgctgactacccagacgagctgaagtgcctggatgctccggtgctgacccaggctgagtgtaaagcctcctaccctggaaagattaccaacagcatgttctgtgtgggcttccttgagggaggcaaggattcctgccagcgtgactctggtggccctgtggtctgcaacggacagctccaaggagttgtctcctggggccatggctgtgcctggaagaacaggcctggagtctacaccaaggtctacaactatgtggactggattaaggacaccatcgctgccaacagctaaagcccccggtccctctgcagtctctataccaataaagtggccctgctctcaaaaaaaaaaaaaaaa

SEQ ID NO: 4 - protein, 261 aa, Homo sapiens
GLELHPLLGGRTWRAARDADGCEALGTVAVPFDDDDKIVGGYTCEENSLPYQVSLNSGSHFCGGSLISEQWVVSAAHCYKTRIQVRLGEHNIKVLEGNEQFINAAKIIRHPKYNRDTLDNDIMLIKLSSPAVINARVSTISLPTAPPAAGTECLISGWGNTLSFGADYPDELKCLDAPVLTQAECKASYPGKITNSMFCVGFLEGGKDSCQRDSGGPVVCNGQLQGVVSWGHGCAWKNRPGVYTKVYNYVDWIKDTIAANS

SEQ ID NO: 5 - protein, 224 aa, Homo sapiens
IVGGYTCEENSLPYQVSLNSGSHFCGGSLISEQWVVSAAHCYKTRIQVRLGEHNIKVLEGNEQFINAAKIIRHPKYNRDTLDNDIMLIKLSSPAVINARVSTISLPTAPPAAGTECLISGWGNTLSFGADYPDELKCLDAPVLTQAECKASYPGKITNSMFCVGFLEGGKDSCQRDSGGPVVCNGQLQGVVSWGHGCAWKNRPGVYTKVYNYVDWIKDTIAANS

SEQ ID NO: 6 - protein, 58 aa, Homo sapiens
TEDYCLASNKVGRCRGSFPRWYYDPTEQICKSFVYGGCLGNKNNYLREEECILACRGV

SEQ ID NO: 7 - protein, 58 aa, Homo sapiens
IHDFCLVSKVVGRCRASMPRWWYNVTDGSCQLFVYGGCDGNSNNYLTKEECLKKCATV

SEQ ID NO: 8 - protein, 58 aa, Homo sapiens
YEEYCTANAVTGPCRASFPRWYFDVERNSCNNFIYGGCRGNKNSYRSEEACMLRCFRQ

SEQ ID NO: 9 - protein, 58 aa, Homo sapiens
MHSFCAFKADDGPCKAIMKRFFFNIFTRQCEEFIYGGCEGNQNRFESLEECKKMCTRD

SEQ ID NO: 10 - protein, 58 aa, Homo sapiens
KPDFCFLEEDPGICRGYITRYFYNNQTKQCERFKYGGCLGNMNNFETLEECKNICEDG

SEQ ID NO: 11 - protein, 58 aa, Homo sapiens
NAEICLLPLDYGPCRALLLRYYYDRYTQSCRQFLYGGCEGNANNFYTWEACDDACWRI

SEQ ID NO: 12 - protein, 59 aa, Homo sapiens
TVAACANLPIVRGPCRAFIQLWAFDAVKGKCVLFPYGGCQGNGNKFYSEKECREYCGVP

SEQ ID NO: 13 - protein, 58 aa, Homo sapiens
VKAVCSQEAMTGPCRAVMPRWYFDLSKGKCVRFIYGGCGGNRNNFESEDYCMAVCKAM

(19) United States
(12) Patent Application Publication
(10) Pub. No.: US 2012/0094923 A1
(43) Pub. Date: Apr. 19, 2012

(54) METHODS AND MATERIALS FOR ASSESSING PROSTATE CANCER RECURRENCE AND REDUCING MESOTRYPSIN ACTIVITY

(76) Inventors: Evette S. Radisky, Jacksonville, FL (US); Mohd A. Salameh, Jacksonville, FL (US)

(21) Appl. No.: 13/270,980

(22) Filed: Oct. 11, 2011

Related U.S. Application Data
(60) Provisional application No. 61/392,894, filed on Oct. 13, 2010.

(51) Int. Cl. A61K 38/57; G01N 33/566; A61P 35/00; C12Q 1/37; C12N 9/99; C12Q 1/68

(52) U.S. Cl. 514/19.3; 435/184; 435/6.12; 435/7.92; 435/23; 436/501

(57) ABSTRACT

This document provides methods and materials involved in assessing prostate cancer recurrence and reducing mesotrypsin activity. For example, methods and materials for using expression levels of PRSS3 nucleic acid to determine whether a prostate cancer patient having undergone prostatectomy is likely to experience prostate cancer recurrence are provided. In addition, methods and materials for reducing mesotrypsin activity are provided.

### CROSS-REFERENCE TO RELATED APPLICATIONS

This application claims the benefit of U.S. Provisional Application Ser. No. 61/392,894, filed Oct. 13, 2010. The disclosure of the prior application is considered part of (and is incorporated by reference in) the disclosure of this application.

### STATEMENT AS TO FEDERALLY SPONSORED RESEARCH

This invention was made with government support under grant CA091956 awarded by the National Institutes of Health. The government has certain rights in the invention.

### BACKGROUND

1. Technical Field

This document relates to methods and materials involved in assessing prostate cancer recurrence and reducing mesotrypsin activity. For example, this document provides methods and materials for using expression levels of PRSS3 nucleic acid to determine whether a prostate cancer patient having undergone prostatectomy is likely to experience prostate cancer recurrence. This document also provides methods and materials for reducing mesotrypsin activity.

2. Background Information

The prostate is a gland in the male reproductive system located below the bladder and in front of the rectum. The main function of the prostate gland, which is about the size of a walnut, is to make fluid for semen. Prostate cancer occurs when a malignant tumor forms in the tissue of the prostate. Although there are several cell types in the prostate, nearly all prostate cancers start in the gland cells. This type of cancer is known as adenocarcinoma.

Prostate cancer is the second leading cause of cancer-related death in American men. Most of the time, prostate cancer grows slowly. Autopsy studies show that many older men who died of other diseases also had prostate cancer that neither they nor their doctor were aware of Sometimes, however, prostate cancer can grow and spread quickly. When localized to the prostate, treatments are delivered with curative intent, either with surgical prostatectomy or radiation.

### SUMMARY

This document provides methods and materials involved in assessing prostate cancer recurrence and reducing mesotrypsin activity. For example, this document provides methods and materials for using expression levels of PRSS3 nucleic acid to assess prostate cancer patients having undergone prostatectomy for an increased likelihood of experiencing prostate cancer recurrence. PRSS3 nucleic acid encodes mesotrypsin, a human trypsin polypeptide. Mesotrypsin is produced and secreted as a larger polypeptide referred to in some cases as a mesotrypsinogen polypeptide, a trypsinogen IV polypeptide, or a trypsinogen 4 polypeptide. As described herein, metastatic prostate tumors exhibit an increased level of PRSS3 nucleic acid expression as compared to benign prostate tumors and clinically localized prostate tumors. Having the ability to identify prostate cancer patients having an increased likelihood of experiencing prostate cancer recurrence can allow clinicians and patients determine proper treatment and medical care options.

This document also provides methods and materials for reducing mesotrypsin activity. For example, this document provides methods and materials for using a mesotrypsin inhibitor to reduce mesotrypsin activity. As described herein, a bovine pancreatic trypsin inhibitor (BPTI)-based molecule (e.g., BPTI-K15R/R17G) can be used as a mesotrypsin inhibitor to reduce mesotrypsin activity. In some cases, a mesotrypsin inhibitor such as a BPTI-based molecule (e.g., BPTI-K15R/R17G) can be administered to a mammal (e.g., a human) to reduce mesotrypsin activity within the mammal

This document also provides methods and materials for reducing cancer cell growth. For example, this document provides methods and materials for using a mesotrypsin inhibitor to reduce cancer cell growth. As described herein, a mesotrypsin inhibitor such as a BPTI-based molecule (e.g., BPTI-K15R/R17G) can be placed into contact with cancer cells to reduce cancer cell growth. In some cases, a mesotrypsin inhibitor such as a BPTI-based molecule (e.g., BPTI-K15R/R17G) can be administered to a mammal (e.g., a human) to reduce cancer cell growth.

In general, one aspect of this document features a method for treating cancer. The method comprises, or consists essentially of, administering a mesotrypsin inhibitor to the mammal, thereby treating the cancer. The mammal can be a human. The cancer can be prostate cancer or breast cancer. The inhibitor can be BPTI-K15R/R17G.

In another aspect, this document features a method for reducing cancer cell growth. The method comprises, or consists essentially of, administering a mesotrypsin inhibitor to a mammal having cancer cells under conditions wherein growth of the cancer cells within the mammal is reduced. The mammal can be a human. The cancer cells can be prostate cancer cells or breast cancer cells. The inhibitor can be BPTI-K15R/R17G.

In another aspect, this document features a method for reducing mesotrypsin activity. The method comprises, or consists essentially of, contacting mesotrypsin with a mesotrypsin inhibitor comprising a Kunitz domain under conditions wherein the activity of the mesotrypsin is reduced, wherein the mesotrypsin inhibitor has a Kᵢ less than 2×10⁻⁵ M and a k_{cat} (s⁻¹) value less than 5×10⁻³. The mesotrypsin inhibitor can have a Kᵢ less than 5×10⁻⁷ M and a k_{cat} (s⁻¹) value less than 1×10⁻³. The mesotrypsin inhibitor can be administered to a mammal to contact the mesotrypsin. The mammal can be a human. The inhibitor can be BPTI-K15R/R17G.

In another aspect, this document features a method for identifying a human having had a prostatectomy as being likely to experience prostate cancer recurrence. The method comprises, or consists essentially of, (a) detecting the presence of an elevated level of PRSS3 nucleic acid expression in the patient, and (b) classifying the human as being likely to experience prostate cancer recurrence based at least in part on the presence of the elevated level of PRSS3 nucleic acid expression.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1. PRSS3 expression in clinical prostate cancer samples. (A) PRSS3 expression correlates with increasing progression. (B) High PRSS3 expression predicts early cancer recurrence following prostatectomy.

FIG. 2. Prostate cancer cell progression and PRSS3 expression. (A) In the RWPE-1-derived progression series, tumorigenic cell lines NB14, NB11, and NB26 display progressively malignant and invasive phenotypes in culture and in xenografts. This figure was adapted from Webber et al. (Prostate, 47(1):1-13 (2001)). (B) PRSS3 transcript levels were assessed by qRT/PCR with expression normalized vs. GAPDH.

FIG. 3. Effects of mesotrypsin on 3D growth morphology and invasion of NB11 cells. Cells grown in Matrigel were treated with buffer only (A) or 100 nM mesotrypsin (B) for 7 days. (C) Untreated or mesotrypsin-treated cells were also evaluated for invasion using BD Biocoat Matrigel invasion chambers.

FIG. 4. PRSS3 knockdown blocks invasion of prostate cancer cells. NB26 cells (A) or PC3-M cells (B) were transduced with a PRSS3-targeted shRNA (kd) or nontarget control (NT) construct; PRSS3 transcript levels were assessed in pooled transductants after puromycin selection by qRT/PCR with expression levels normalized vs. GAPDH. NB26 (C) and PC3-M (D) PRSS3 kd pooled transductants show striking reductions in invasion relative to NT controls in Matrigel transwell invasion assays.

FIG. 5. Prostate cancer cell malignant morphology is induced specifically by active mesotrypsin. NB11 cells were grown in 3D culture for 4 days, untreated (A), or treated with 100 nM recombinant mesotrypsin (B), calatytically inactive mesotrypsin-S195A (C), or cationic trypsin (D).

FIG. 6. COX-2 mediates mesotrypsin-potentiated malignancy. (A) COX-2 expression is induced by mesotrypsin treatment of NB 11 cells, as found by qRT/PCR. (B) Transduction of NB11 cells with a shRNA lentivirus targeting COX2 suppresses COX2 expression, relative to cells transduced with a nontarget (NT) virus, as found by qRT/PCR. (C) Morphological changes induced by mesotrypsin in control cells (top) are blocked in cells transduced with the COX-2 knockdown construct (bottom).

FIG. 7. Upregulation of COX-2-luciferase reporter in response to mesotrypsin. NB11 cells transfected with wt COX-2/luc were treated with buffer only (control) or 100 nM mesotrypsin (PRSS3) when seeding cells into 3D; expression was assessed after two days using the Luciferase Assay System (Promega).

FIG. 8. Lentiviral transduction of PC3-M cells and orthotopic tumor growth in mice. PC3-M cells were transduced with pSIN-luc lentivirus for firefly luciferase expression or with pSIN-CSGW control virus for GFP expression. Comparison of GFP control cells by phase contrast (A) or fluorescence microscopy (B) shows high transduction efficiency. (C) A luciferase assay of pSIN-luc-transduced cell dilutions detects fewer than 20 cells. (D) BLI was used to follow tumor growth in male NOD/SCID mice implanted orthotopically with 100K pSIN-luc-transduced PC3-M cells; weekly images from a representative mouse are shown. (E) Tumor growth as assessed by weekly BLI imaging is plotted for a group of 3 mice.

FIG. 9. Knockdown of PRSS3 in PC3-M cells reduces spontaneous metastasis. PC3-M cells transduced with a lentiviral shRNA targeting PRSS3 (KD) or with a nontarget control virus (NT) were superinfected with pSIN-luc conferring luciferase expression, and implanted orthotopically into mice (100K cells). (A) PRSS3 knockdown showed some evidence for a minimal suppression of tumor growth, as assessed by in vivo BLI. (B) In mice harvested at 2 weeks post-implantation, PRSS3 knockdown resulted in a substantially reduced burden of pulmonary metastases, as assessed by ex vivo BLI at necropsy. Shown also are representative ex vivo BLI images taken from (C) a control mouse, showing extensive metastasis in all lung lobes, and (D) a knockdown mouse, showing limited metastasis in one lung lobe. Lung metastases were confirmed by staining with anti-human pan-cytokeratin clones AE1/AE3 (E), as well as H&E staining (F).

FIG. 10. Comparison of BPTI and APPI sequences (SEQ ID NOs:1 and 2, respectively) and structures. A, A sequence alignment of the two Kunitz inhibitors shows two nonidentical residues (indicated by asterisks) within the canonical binding loop (boxed region): a conservative difference (Lys vs. Arg) at the P₁ position, and a nonconservative difference (Arg vs. Met) at the P₂′ position. Structural features including two β-strands and the C-terminal α-helix are indicated above the corresponding sequences. B, Structural comparisons of BPTI (left) and APPI (right), from crystal structures solved in our laboratory of the two inhibitors in complexes with mesotrypsin, show the overall similarity of the Kunitz inhibitor fold and the conformational similarities within the canonical binding loops of the inhibitors; these loops are responsible for the majority of the inhibitor-enzyme contacts.

FIG. 11. Hydrolysis of APPI and BPTI variants monitored by reversed-phase HPLC. For each inhibitor, first and last sample traces are shown from representative hydrolysis time courses; the x-axis shows retention time in minutes, and the y-axis shows absorbance at 210 nm. A, Separation of intact APPI (peak 1) from cleaved product(s). As samples were not treated with a reducing agent to reduce internal disulfide bonds, the major cleaved product is eluted as a single species (peak 2). A peak was not observed for mesotrypsin in the digests of APPI variants, because mesotrypsin concentration was below the level of detection in these reactions. For the APPI-R15K/M17R variant, the intact inhibitor represented a mixture of two species that were hydrolyzed by mesotrypsin with identical rates. Mass spectrometry and N-terminal sequencing identified the major peak as APPI possessing the expected N-terminal sequence, while the minor peak represented APPI with an additional three amino acids at the N-terminus, possibly due to differences in processing during secretion by Pichia pastoris. B, Separation of intact BPTI (peak 3) from cleaved products. Because samples were reduced with DTT prior to analysis, the N-terminal fragment (peak 4) and C-terminal fragment (peak 5) are resolved, as is mesotrypsin (peak 6). The depletion of intact inhibitors over time was quantified by peak integration, allowing calculation of rates of hydrolysis.

FIG. 12. “Thermodynamic cube” summarizing the additivity of free energy changes attributable to the P₁ residue, P₂′ residue, and scaffold. Each corner of the cube represents a different BPTI or APPI variant, as annotated. Parallel edges of the cube represent the same “mutation” on a different sequence background. The four vertical edges of the cube represent an Arg to Lys mutation at position 15 (P₁), the four horizontal edges of the cube represent a Met to Arg mutation at position 17 (P₂′), and the four diagonal edges represent the composite group of mutations converting the APPI scaffold to the BPTI scaffold. A, Values along each edge represent ΔΔG_{cat} calculated using eq. 3; shaded faces of the cube identify significantly nonadditive cycles. B, Values along each edge represent ΔΔGₐ° calculated using eq. 4; shaded faces of the cube (all faces except the left P₁ vs. scaffold cycle) identify significantly nonadditive cycles. C, Values along each edge represent ΔΔG_{T}^{‡} calculated using eq. 2; shaded faces of the cube identify significantly nonadditive cycles.

FIG. 13. Differences in APPI bound to mesotrypsin vs. bovine trypsin. Structure of the mesotrypsin/APPI complex superposed with the bovine trypsin/APPI complex (PDB ID 1TAW) shows the impact of mesotrypsin Arg-193 (Gly-193 in bovine trypsin) on the positioning of the APPI residues Met-17 and Phe-34.

FIG. 14. Evidence for relative flexibility of primed side APPI residues bound to mesotrypsin. A, Superposed structures of the four copies of the mesotrypsin/APPI complex in the asymmetric unit show conformational heterogeneity of Met-17. B, Crystallographic B-factors for backbone atoms (N, C_{α}, C, and O) are plotted as a function of residue number for the canonical loop of APPI (chain E) complexed with mesotrypsin (circles), APPI (chain B) complexed with bovine trypsin (squares; PDB ID 1TAW), and BPTI (chain E) complexed with mesotrypsin (triangles, PBD ID 2R9P). For reference, the mesotrypsin/APPI structure is 2.5 Å resolution with average protein B=46.1, the bovine trypsin/APPI structure is 1.8 Å resolution with average protein B=34.0, and the mesotrypsin/BPTI structure is 1.4 Å resolution with average protein B=22.6.

FIG. 15. Differential interactions of APPI-wt P₁ Arg and APPI-R15K P₁ Lys in the mesotrypsin S₁ specificity pocket. A, Mesotrypsin is shown in light gray, with Arg-15 of APPI-wt in black. Arg-15 forms direct H-bonding interactions with Asp-189 and Ser-190 side chains, with the Glu-219 carbonyl oxygen, and with one water molecule. B, Mesotrypsin is shown in light gray, with Lys-15 of APPI-R15K in white. Lys-15 N_{ζ} lies within direct H-bonding distance of Ser-190 side chain and carbonyl oxygens, as well as two water molecules, one of which bridges an interaction with Asp-189 O_{δ2}. Direct interaction with Asp-189 O_{δ1} may also provide electrostatic stabilization, although the interatomic distance is longer at 3.79 Å.

FIG. 16. Mesotrypsin promotes spontaneous metastasis in orthotopic xenograft model. (A) Tumors from three animals of the test group, in which implanted PC3-M cells were transduced with an shRNA virus targeting PRSS3, exhibited persistent suppression of PRSS3 expression relative to a tumor from a control animal (NT). (B) PRSS3 knockdown resulted in significantly reduced burden of pulmonary metastases as assessed by ex vivo bioluminescent imaging at necropsy (*, P=0.0021). (C) Ex vivo bioluminescent image of heart and lungs from a control mouse with flux measurement near the group median (left) revealed extensive metastasis in all lung lobes, by contrast with image of heart and lungs from a representative test group mouse (right), which revealed no evidence of metastasis.

FIG. 17. Mesotrypsin inhibition reduces prostate cancer cell invasion. (A) PC3-M prostate cancer cells were transduced with a lentiviral shRNA construct specifically targeting PRSS3 (KD), or with a non-target control virus (NT). Measurement of PRSS3 transcript levels by qRT/PCR, normalized to GAPDH, confirmed efficient knockdown of PRSS3. (B) In Matrigel transwell invasion assays, cells with shRNA knockdown of PRSS3, as well as cells assayed in the presence of 100 nM BPTI-K15R/R17G mesotrypsin inhibitor, showed significantly reduced invasion. Photographs of representative fields from invasion filters are shown above the graphical results. Error bars represent SEM for triplicate assay wells (*, P<0.01).

FIG. 18. Mesotrypsin inhibition reduces malignant growth of breast cancer cells in 3D culture. HMT3522 T4-2 cells were grown in Matrigel for 7 days in the presence of the indicated concentrations of BPTI-wt or BPTI-K15R/R17G inhibitors, then photographed and assessed for colony size. (A) Photographs of representative fields show an inhibitor concentration-dependent reduction of colony size compared to control cultures; size bar, 100 μm. (B) Colony size (obtained from morphometric measurement of 30 colonies per condition) was significantly reduced by both inhibitors; however, the BPTI-K15R/R17G mutant was ˜10-fold more potent, as colony growth with 100 nM BPTI-K15R/R17G was similar to that observed in cultures treated with 1000 nM BPTI-wt.

FIG. 19 is a listing of a nucleic acid sequence (SEQ ID NO:3) of a human PRSS3 cDNA (GenBank Accession No. X72781.1; GI No. 3928429).

FIG. 20 is a listing of amino acid sequences of (a) a human trypsinogen IV polypeptide (SEQ ID NO:4), and (b) a human mesotrypsin polypeptide (SEQ ID NO:5).

FIG. 21 is a sequence alignment of BPTI, APPI, a hepatocyte growth factor activator inhibitor-1 (HAI-1) Kunitz domain 1 (SEQ ID NO:6), a hepatocyte growth factor activator inhibitor-2 (HAI-2) Kunitz domain 1 (SEQ ID NO:7), an HAI-2 Kunitz domain 2 (SEQ ID NO:8), a tissue factor pathway inhibitor-1 (TFPI-1) Kunitz domain 1 (SEQ ID NO:9), a TFPI-1 Kunitz domain 2 (SEQ ID NO:10), a tissue factor pathway inhibitor-2 (TFPI-2) Kunitz domain 1 (SEQ ID NO:11), a bikunin Kunitz domain 2 (SEQ ID NO:12), and an amyloid precursor-like protein 2 (APLP2) Kunitz domain (SEQ ID NO:13). The five-residue stretch representing the P₁, P₁′, P₂′, P₃′, and P₄^{′} residues, as well as the isolated residue 34, are shown in bold font and boxed.

FIG. 22. Impact of P₂′ side chain size on free energies of association, catalysis, and transition state stabilization for mesotrypsin interaction with APPI. The number of non-hydrogen atoms in the P₂′ side chain was plotted vs. the change in free energy of association relative to P₂′ Gly ΔΔGₐ° (Gly17X) (A), the change in free energy of catalysis ΔΔG_{cat}(Gly17X) (B), and the change in the transition-state stabilization energy ΔΔG_{T}^{‡}(Gly17X) (C). P₂′ residues associated with each data point are indicated using the one-letter code.

FIG. 23. Distinct conformations of mesotrypsin Arg-193 shaped by alternative P₂′ residues of bound BPTI. Crystal structures of mesotrypsin in complex with BPTI-K15R/R17G (A), BPTI-WT (B), and BPTI-K15R/R17D (C) revealed globally similar complexes (left panels) with significant differences in interface topology in the vicinity of Arg-193 (surface shown in color). (A) In the BPTI-K15R/R17G complex, Arg-193 extended downward, enveloping BPTI Gly-17. In the BPTI-WT complex, Arg-193 receded into a crevice on the surface of the enzyme (B), while a distinct intermediate conformation of Arg-193 was found in the BPTI-K15R/R17D complex (C). 2Fo-Fc density maps (contoured at 1.5 sigma) revealed well-ordered side chains for Arg-193 in the BPTI-K15R/R17G complex (D) and for both Arg-193 and the P₂′ Arg in the BPTI-WT complex (E). Arg-193 and the P₂′ Asp side chain were less well defined in the BPTI-K15R/R17D complex despite the higher resolution of this structure (F).

FIG. 24. Conformational changes of mesotrypsin upon inhibitor binding. Superposition of mesotrypsin•BPTI structures with the mesotrypsin•benzamidine structure 1H4W (mesotrypsin beige, benzamidine red), in which the primed-side subsites are unfilled, revealed the conformational rearrangements required of mesotrypsin Arg-193 upon BPTI binding. Only minor adjustments of Arg-193 were observed in the mesotrypsin•BPTI-K15R/R17G complex (A), whereas Arg-193 was shifted upward by ˜6 Å in the mesotrypsin•BPTI-WT structure (B) and by ˜3.5 Å in the mesotrypsin•BPTI-K15R/R17D structure (C).

FIG. 25. Effect of BPTI-K15R/R17G on invasion of pancreatic cancer cells. Transduction of Capan-1 cells with an shRNA construct specifically targeting PRSS3 resulted in suppression of expression at the transcript level as assessed by qRT/PCR (A), and at the protein level as assessed by Western blot (B). (C) In Matrigel transwell invasion assays, Capan-1 cells assayed in the presence of 100 nM BPTI-K15R/R17G (NT+I) showed reduced invasion relative to control cells (NT). 100 nM inhibitor gave results similar to shRNA knockdown of PRSS3 (KD). The graph provides mean and SEM for quadruplicate membranes. Representative fields from invasion filters are shown above graphical results. *, p<0.05; ***, p<0.0005 (unpaired t test).

### DETAILED DESCRIPTION

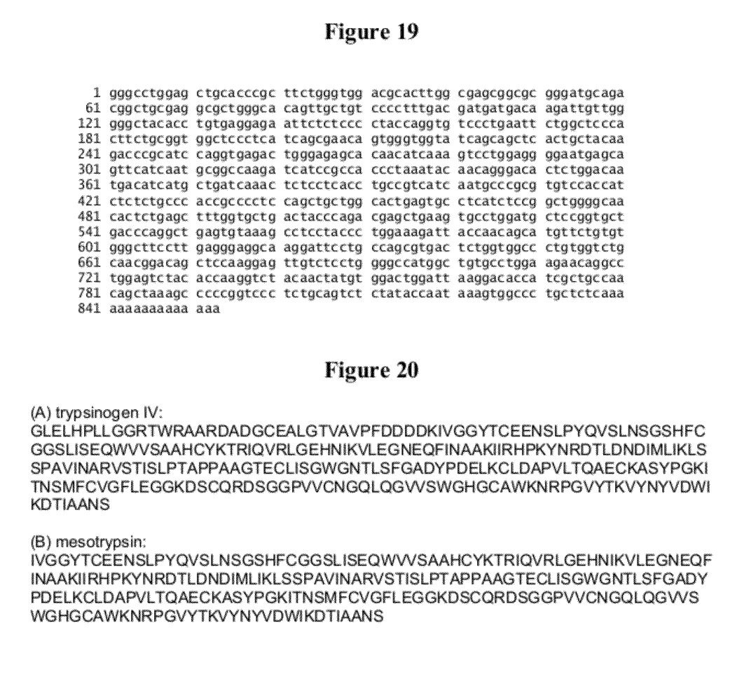

This document provides methods and materials involved in assessing prostate cancer recurrence and reducing mesotrypsin activity. For example, this document provides methods and materials for using expression levels of PRSS3 nucleic acid to assess prostate cancer patients having undergone prostatectomy for an increased likelihood of experiencing prostate cancer recurrence. As described herein, metastatic prostate tumors exhibit an increased level of PRSS3 nucleic acid expression as compared to benign prostate tumors and clinically localized prostate tumors. A human PRSS3 nucleic acid can have the nucleic acid sequence set forth in FIG. 19 and can encode a trypsinogen IV or mesotrypsin polypeptide having the amino acid sequence set forth in FIG. 20.

The term “elevated level” as used herein with respect to the level of PRSS3 nucleic acid expression is any level that is above a median level of PRSS3 nucleic acid expression in benign prostate tissue. Any appropriate method can be used to determine the level of PRSS3 nucleic acid or trypsinogen IV/mesotrypsin polypeptide expression in a sample (e.g., prostate tissue sample) from a mammal For example, mass spectrometry, FACS, Western blotting, ELISA, immunohistochemistry, and immunoprecipitation can be used to determine the level of mesotrypsin in a sample. In some cases, RT-PCR techniques and other nucleic acid-based assays can be used to determine the level of PRSS3 nucleic acid expression in a sample.

Once the level of PRSS3 nucleic acid expression is determined, then the level can be compared to a median level or a cutoff level and used to determine whether or not the mammal is likely to experience prostate cancer recurrence. If it is determined that a sample from a mammal contains an elevated level of PRSS3 nucleic acid expression, then the mammal can be classified as being likely to experience prostate cancer recurrence. In some cases, the level of PRSS3 nucleic acid expression in a sample can be used in combination with one or more other factors to determine whether or not a mammal is likely to experience prostate cancer recurrence.

This document also provides methods and materials to assist medical or research professionals in determining whether or not a mammal is likely to experience prostate cancer recurrence. Medical professionals can be, for example, doctors, nurses, medical laboratory technologists, and pharmacists. Research professionals can be, for example, principle investigators, research technicians, postdoctoral trainees, and graduate students. A professional can be assisted by (1) determining the level of PRSS3 nucleic acid expression in a sample, and (2) communicating information about the level to that professional.

Any appropriate method can be used to communicate information to another person (e.g., a professional). For example, information can be given directly or indirectly to a professional. In addition, any type of communication can be used to communicate the information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In addition, the information can be communicated to a hospital, clinic, or research facility serving as an agent for the professional.

This document also provides methods and materials related to treating conditions that involve mesotrypsin activity. Examples of such conditions include, without limitation, cancer, multiple sclerosis, and pancreatitis. For example, this document provides methods and materials for reducing cancer cell growth (e.g., prostate cancer cell, breast cancer cell, colon cancer cell, or lung cancer cell growth). As described herein, an inhibitor of mesotrypsin activity can be used to reduce cancer cell growth.

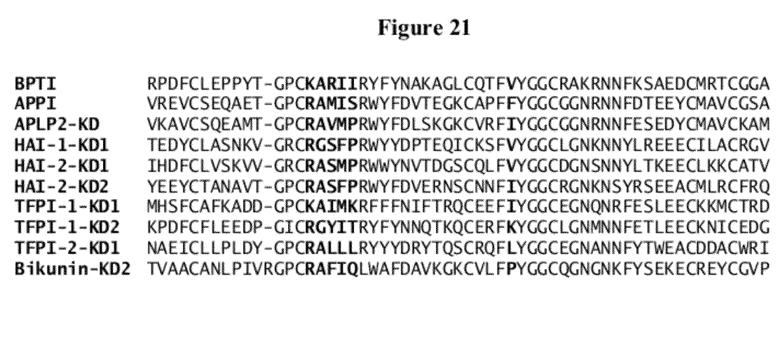

Any type of mammal having a condition that involves mesotrypsin activity can be treated as described herein. For example, humans and other primates such as monkeys having prostate or breast cancer can be treated with one or more mesotrypsin inhibitors. In some cases, dogs, cats, horses, cows, pigs, sheep, mice, and rats can be treated with one or more mesotrypsin inhibitors as described herein. Any appropriate method can be used to identify mesotrypsin inhibitors. For example, the methods described herein can be used to identify mesotrypsin inhibitors. Examples of mesotrypsin inhibitors include, without limitation, siRNA and shRNA molecules designed to reduce PRSS3 nucleic acid expression, antisense molecules, BPTI molecules, BPTI-based molecules such as BPTI-K15R/R17G, and polypeptide molecules having one or more Kunitz domains such as HAI-1 molecules, HAI-1-based molecules, HAI-2 molecules, HAI-2-based molecules, TFPI-1 molecules, TFPI-1-based molecules, TFPI-2 molecules, TFPI-2-based molecules, bikunin Kunitz domain 2 molecules, bikunin Kunitz domain 2-based molecules, APLP2 molecules, and APLP2-based molecules. In some cases, a molecule presented in Table 7 or FIG. 21 or a molecule presented in Table 7 or FIG. 21 having one or more (e.g., one, two, three, four, five, six, or more) amino acid substitutions can be used as a mesotrypsin inhibitor as described herein.

A mesotrypsin inhibitor provided herein (e.g., a BPTI-based molecule such as BPTI-K15R/R17G) can have a Kᵢ of less than 2×10⁻⁵ M (e.g., less than 1×10⁻⁵ M, less than 5×10⁻⁶ M, less than 1×10⁻⁶ M, less than 9×10⁻⁷ M, less than 8×10⁻⁷ M, less than 7×10⁻⁷ M, less than 6×10⁻⁷ M, less than 5×10⁻⁷ M, less than 4×10⁻⁷ M, less than 3×10⁻⁷ M, less than 2×10⁻⁷ M, less than 1×10⁻⁷ M, or less than 5×10⁻⁸M) and can have a k_{cat} (s⁻¹) value less than 9×10⁻³ (e.g., less than 8×10⁻³, less than 7×10⁻³, less than 6×10⁻³, less than 5×10⁻³, less than 4×10⁻³, less than 3×10⁻³, less than 2×10⁻³, less than 1×10⁻³, less than 7.5×10⁻⁴, less than 5×10⁻⁴, less than 2.5×10⁻⁴, or less than 1×10⁻⁴). Any appropriate method can be used to determine if a mesotrypsin inhibitor has a Kᵢ of less than 2×10⁻⁵ M (e.g., less than 1×10⁻⁵ M, less than 5×10⁻⁶ M, less than 1×10⁻⁶M, less than 9×10⁻⁷ M, less than 8×10⁻⁷ M, less than 7×10⁻⁷ M, less than 6×10⁻⁷ M, less than 5×10⁻⁷M, less than 4×10⁻⁷ M, less than 3×10⁻⁷M, less than 2×10⁻⁷ M, less than 1×10⁻⁷ M, or less than 5×10⁻⁸ M). For example, the methods described herein can be used to determine if a mesotrypsin inhibitor has a Kᵢ of less than 2×10⁻⁵ M. Any appropriate method can be used to determine if a mesotrypsin inhibitor has a k_{cat} (s⁻¹) value less than 9×10⁻³ (e.g., less than 8×10⁻³, less than 7×10⁻³, less than 6×10⁻³, less than 5×10⁻³, less than 4×10⁻³, less than 3×10⁻³, less than 2×10⁻³, less than 1×10⁻³, less than 7.5×10⁻⁴, less than 5×10⁻⁴, less than 2.5×10⁻⁴, or less than 1×10⁻⁴). For example, the methods described herein can be used to determine if a mesotrypsin inhibitor has a k_{cat} (s⁻¹) value less than 9×10⁻³.

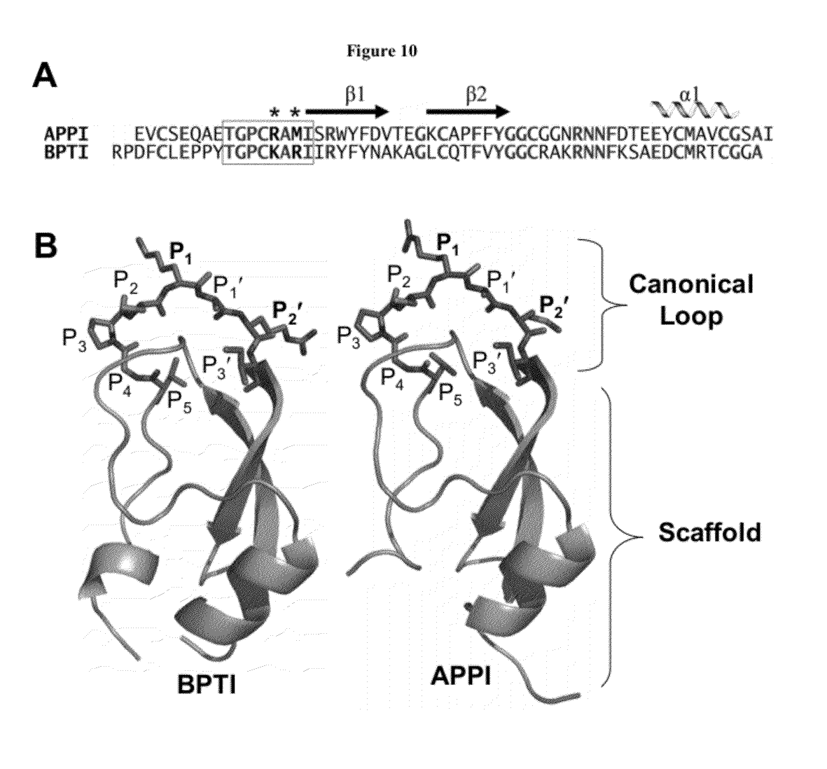

As described herein, polypeptide molecules having one or more Kunitz domains can be engineered to have reduced Kᵢ and reduced k_{cat} (s⁻¹) values. For example, a BPTI-based molecule can be engineered to contain an amino acid substitution at one or more one or more (e.g., one, two, three, four, five, six, or more) positions such as the P1, P1′, P2′, P3′, P4′, and residue 34 positions. See, e.g., FIGS. 10 and 21. In some cases, an HAI-1-based molecule can have an HAI-1 sequence with an amino acid substitution at one or more positions (e.g., the P1, P1′, P2′, P3′, P4′, and residue 34 positions), an HAI-2-based molecule can have an HAI-2 sequence with an amino acid substitution at one or more positions (e.g., the P1, P1′, P2′, P3′, P4′, and residue 34 positions), TFPI-1-based molecule can have a TFPI-1 sequence with an amino acid substitution at one or more positions (e.g., the P1, P1′, P2′, P3′, P4′, and residue 34 positions), TFPI-2-based molecule can have a TFPI-2 sequence with an amino acid substitution at one or more positions (e.g., the P1, P1′, P2′, P3′, P4′, and residue 34 positions), a bikunin Kunitz domain 2-based molecule can have a bikunin Kunitz domain 2 sequence with an amino acid substitution at one or more positions (e.g., the P1, P1′, P2′, P3′, P4′, and residue 34 positions), and an APLP2-based molecule can have an APLP2 sequence with an amino acid substitution at one or more positions (e.g., the P1, P1′, P2′, P3′, P4′, and residue 34 positions). See, e.g., FIGS. 10 and 21. For example, a polypeptide molecule having one or more Kunitz domains can be designed to have an Arg at the P1 position and a Gly or Ala at the P2′ position. In some cases, a polypeptide molecule having one or more Kunitz domains can be designed to have a Ser or Thr residue at the P1′ position.

In some cases, a polypeptide molecule having one or more Kunitz domains can be designed to have a conservative amino acid substitution or a non-conservative amino acid substitution. Conservative amino acid substitutions can be, for example, aspartic-glutamic as acidic amino acids; lysine/arginine/histidine as basic amino acids; leucine/isoleucine, methionine/valine, alanine/valine as hydrophobic amino acids; serine/glycine/alanine/threonine as hydrophilic amino acids. Conservative amino acid substitutions also include groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. After making an amino acid substitution, the activities of the polypeptide containing the amino acid substitution can be assessed using the assays described herein.

In some cases, a mesotrypsin inhibitor provided herein can include components such as polysaccharides, lipids, acetyl groups, PEG, and/or methyl groups. For example, a BPTI-based molecule such as BPTI-K15R/R17G can be PEGylated or otherwise covalently modified to enhance pharmacokinetics and/or bioavailability.

In some cases, one or more than one mesotrypsin inhibitor (e.g., two, three, four, five, or more mesotrypsin inhibitors) can be administered to a mammal to treat a condition that involves mesotrypsin activity. In some cases, one or more mesotrypsin inhibitors can be formulated into a pharmaceutically acceptable composition. For example, a therapeutically effective amount of BPTI-K15R/R17G can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules.

Pharmaceutically acceptable carriers, adjuvants and vehicles that may be used in the pharmaceutical compositions described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. If required, the solubility and bioavailability of a mesotrypsin inhibitor in a pharmaceutical composition can be enhanced using lipid excipients and/or block copolymers of ethylene oxide and propylene oxide. See, e.g., U.S. Pat. No. 7,014,866 and U.S. Patent Publication Nos. 20060094744 and 20060079502.

A pharmaceutical composition described herein can be designed for oral or parenteral (including subcutaneous, intramuscular, intravenous, and intradermal) administration. Compositions suitable for parenteral administration include aqueous and non-aqueous sterile injection solutions that can contain anti-oxidants, buffers, bacteriostats, and solutes which render the formulation isotonic with the blood of the intended recipient; and aqueous and non-aqueous sterile suspensions which may include suspending agents and thickening agents. The formulations can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

Such injection solutions can be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation can be a sterile injectable solution or suspension in a non-toxic parenterally-acceptable diluent or solvent, for example, as a solution in 1, 3-butanediol. Among the acceptable vehicles and solvents that can be used are mannitol, water, Ringer's solution, and isotonic sodium chloride solution. In addition, sterile, fixed oils are conventionally employed as a solvent or suspending medium. For this purpose, any bland fixed oil can be used including synthetic mono- or di-glycerides. Fatty acids, such as oleic acid and its glyceride derivatives can be used in the preparation of injectables, as can natural pharmaceutically-acceptable oils, such as olive oil or castor oil, including those in their polyoxyethylated versions. These oil solutions or suspensions can contain a long-chain alcohol diluent or dispersant.

In some cases, a pharmaceutically acceptable composition including one or more mesotrypsin inhibitors can be administered locally or systemically. For example, a composition containing BPTI-K15R/R17G can be injected into cancer tissue or can be administered systemically to a mammal (e.g., a human). In some cases, a mesotrypsin inhibitor or a combination of mesotrypsin inhibitors can be administered by different routes. For example, BPTI-K15R/R17G can be administered both orally and by injection. In some cases, one mesotrypsin inhibitor can be administered orally and a second mesotrypsin inhibitor can be administered via injection.

Before administering a composition provided herein (e.g., a composition containing one or more mesotrypsin inhibitors) to a mammal, the mammal can be assessed to determine whether or not the mammal has a condition that involves mesotrypsin activity. Any appropriate method can be used to determine whether or not a mammal has a condition that involves mesotrypsin activity. For example, a mammal (e.g., human) can be identified as having a condition that involves mesotrypsin activity such as prostate cancer or breast cancer using standard diagnostic techniques such as ultrasounds, helical CT, magnetic resonance imaging, and endoscopic ultrasonography.

After identifying a mammal as having a condition that involves mesotrypsin activity, the mammal can be administered a composition containing one or more mesotrypsin inhibitors. A composition containing one or more mesotrypsin inhibitors can be administered to a mammal in any amount, at any frequency, and for any duration effective to achieve a desired outcome (e.g., to reduce a symptom of cancer, to increase survival time, to reduce cancer cell transformed growth and invasion, to reduce tumor cell proliferation, to reduce tumor angiogenesis, and/or to prevent or limit metastasis). In some cases, a composition containing one or more mesotrypsin inhibitors can be administered to a mammal having cancer to reduce tumor cell proliferation and to reduce tumor angiogenesis and metastasis.

Effective doses can vary, as recognized by those skilled in the art, depending on the severity of the condition (e.g., cancer), the route of administration, the sex, age and general health condition of the subject, excipient usage, the possibility of co-usage with other therapeutic treatments such as use of other agents and the judgment of the treating physician.

An effective amount of a composition containing one or more mesotrypsin inhibitors can be any amount that reduces the severity of a symptom of a condition being treated (e.g., cancer) without producing significant toxicity to the mammal For example, an effective amount of a mesotrypsin inhibitor such as BPTI-K15R/R17G can be from about 0.5 mg/kg to about 80 mg/kg (e.g., from about 0.5 mg/kg to about 70 mg/kg, from about 0.5 mg/kg to about 60 mg/kg, from about 0.5 mg/kg to about 50 mg/kg, from about 0.5 mg/kg to about 40 mg/kg, from about 0.5 mg/kg to about 30 mg/kg, from about 0.5 mg/kg to about 20 mg/kg, from about 0.5 mg/kg to about 10 mg/kg, from about 0.5 mg/kg to about 5 mg/kg, from about 0.5 mg/kg to about 1 mg/kg, from about 0.75 mg/kg to about 10 mg/kg, from about 1 mg/kg to about 10 mg/kg, or from about 2 mg/kg to about 10 mg/kg). In some cases, between about 20 mg and 125 mg (e.g., between about 20 mg and 100 mg, between about 20 mg and 90 mg, between about 20 mg and 80 mg, between about 30 mg and 100 mg, between about 40 mg and 100 mg, between about 40 mg and 90 mg, between about 40 mg and 80 mg, or between about 70 mg and 80 mg) of a mesotrypsin inhibitor such as BPTI-K15R/R17G can be administered to an average sized human (e.g., about 70 kg human) once a week for two to 20 weeks. For example, about 75 mg of a mesotrypsin inhibitor such as BPTI-K15R/R17G can be administered to an average sized human (e.g., about 70 kg human) once a week for 12 weeks. If a particular mammal fails to respond to a particular amount, then the amount of mesotrypsin inhibitor can be increased by, for example, two fold. After receiving this higher amount, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition (e.g., cancer) may require an increase or decrease in the actual effective amount administered.

The frequency of administration can be any frequency that reduces the severity of a symptom of a condition to be treated (e.g., cancer) without producing significant toxicity to the mammal For example, the frequency of administration can be from about once a week to about three times a day, or from about twice a month to about six times a day, or from about twice a week to about once a day. The frequency of administration can remain constant or can be variable during the duration of treatment. A course of treatment with a composition containing one or more mesotrypsin inhibitors can include rest periods. For example, a composition containing one or more mesotrypsin inhibitors can be administered daily over a two week period followed by a two week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition (e.g., cancer) may require an increase or decrease in administration frequency.

An effective duration for administering a composition containing one or more mesotrypsin inhibitors can be any duration that reduces the severity of a symptom of the condition to be treated (e.g., cancer) without producing significant toxicity to the mammal Thus, the effective duration can vary from several days to several weeks, months, or years. In general, the effective duration for the treatment of cancer can range in duration from several weeks to several months. In some cases, an effective duration can be for as long as an individual mammal is alive. Multiple factors can influence the actual effective duration used for a particular treatment. For example, an effective duration can vary with the frequency of administration, effective amount, use of multiple treatment agents, route of administration, and severity of the condition being treated.

In certain instances, a course of treatment and the severity of one or more symptoms related to the condition being treated can be monitored. Any method can be used to determine whether or not the severity of a symptom is reduced. For example, the severity of a symptom can be assessed using imaging techniques at different time points.

The invention will be further described in the following examples, which do not limit the scope of the invention described in the claims.

### EXAMPLES

### Example 1

### Mesotrypsin is Upregulated in Advanced Prostate Cancer and is Predictive of Recurrence

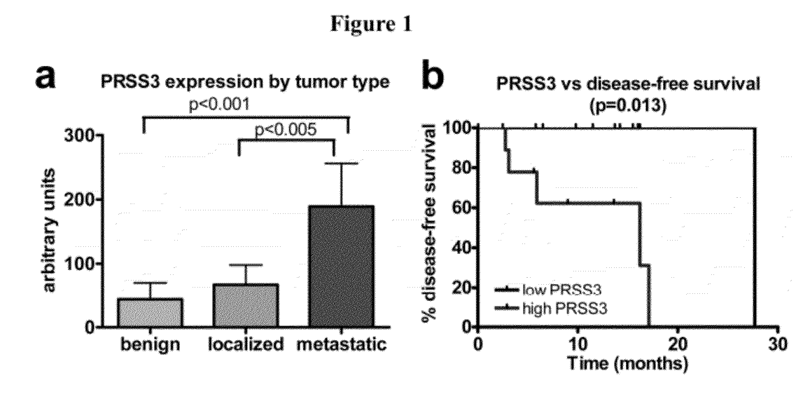

A correlation was identified between PRSS3 expression and prostate cancer progression using a publicly-available microarray dataset (Varambally et al., Cancer Cell, 8(5):393-406 (2005)) in which benign prostate, clinically localized prostate cancer, and metastatic prostate cancer tissue specimens were transcriptionally profiled using Affymetrix U133 2.0 Plus microarrays. PRSS3 expression increased with progression, and significant upregulation of PRSS3 expression was observed in metastatic tumors as compared to benign and clinically localized tumors (FIG. 1A). Data were analyzed from a second open source microarray study which employed Stanford cDNA microarrays, from which clinical follow-up data were available for prostate cancer patients following prostatectomy to remove localized primary prostate adenocarcinomas (Lapointe et al., Proc. Nat'l. Acad. Sci. USA, 101(3):811-816 (2004)). The patients were divided into groups representing upper- and lower-half PRSS3 transcript levels, and Kap-lan-Meier survival analysis was performed. In this cohort, PRSS3 expression in tumors was strikingly predictive of recurrence (FIG. 1B). The association of PRSS3 with prostate cancer metastasis and the evidence that PRSS3 expression in primary tumors is predictive of recurrence indicates that mesotrypsin may promote prostate cancer progression.

### Example 2

### Mesotrypsin Promotes Prostate Cancer Malignant Morphology, Invasion, and Metastasis

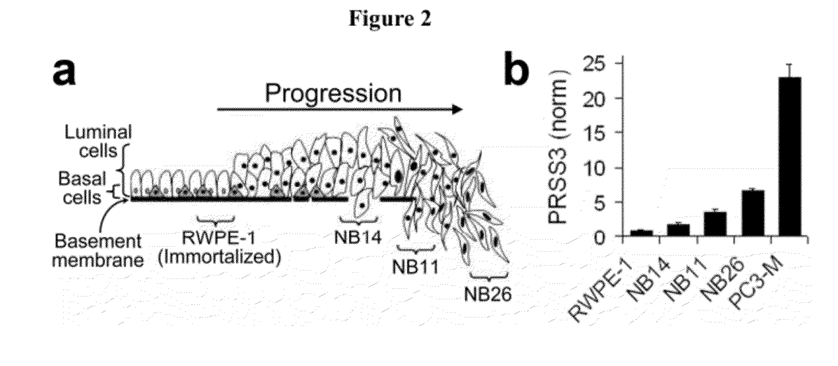

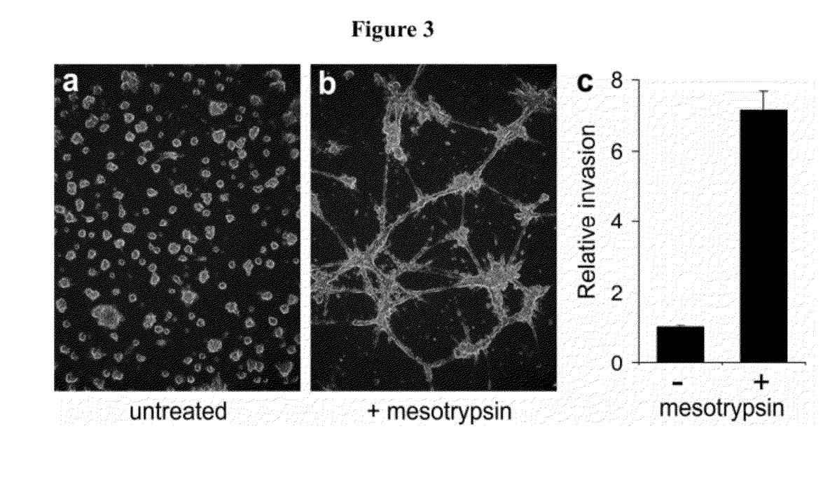

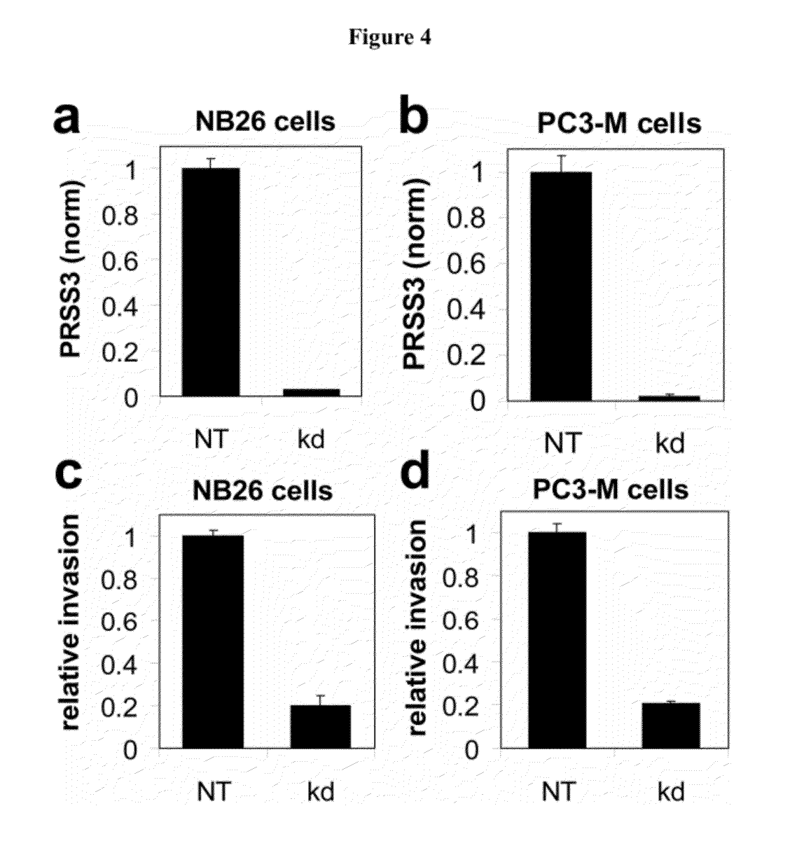

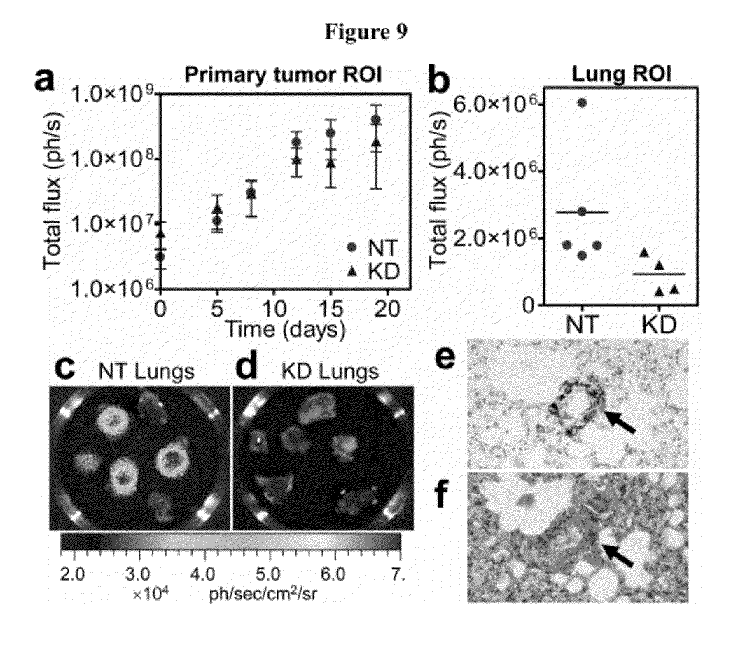

RWPE-1 human prostate cells and the carcinogen-induced derivative cell lines NB14, NB11, and NB26 represent a prostate cancer progression series sharing a common genetic background but featuring progressive phenotypic changes that model prostate cancer progression from pre-neoplastic epithelial cells to prostatic intra-epithelial neoplasia (PIN) to invasive prostatic carcinoma (FIG. 2A). The series exhibited a progression toward malignancy with regard to loss of cell polarity and markers of differentiation, the acquisition of invasive behavior, tumorigenicity in xenografts, and the histopathological grade of resultant tumors. This series provided a tractable model for the study of prostate cancer progression and can be used to assay the effectiveness of prostate cancer interventions at different stages of progression, both in cell culture and in xenografts. Using qRT/PCR to assess transcript levels of PRSS3, nonmalignant RWPE-1 progenitor cells were found to express very low levels of PRSS3, while the carcinogen-transformed cell lines NB14, NB11, and NB26 exhibited increasing levels of mesotrypsin expression that correlated with increasing malignancy (FIG. 2B), consistent with the pattern seen in human tumor samples (FIG. 1A). Cell lines of this series were used to study the effects of mesotrypsin on tumor cells. To determine whether active mesotrypsin plays a functional role in promoting prostate cancer malignant progression, 3D Matrigel culture assays were used to study cell growth morphology in a physiologically relevant environment. While untreated cells grew as small clusters in 3D as described elsewhere (Webber et al., Carcinogenesis, 18(6):1225-1231 (1997); see, also FIG. 3A), treatment with purified recombinant mesotrypsin was found to stimulate an altered growth phenotype characterized by a web-like branching morphology (FIG. 3B). Because the branching morphology observed in mesotrypsin-treated cultures was suggestive of increased invasion through the reconstituted basement membrane, invasion was quantified using Matrigel transwell assays (Zhang et al., J. Biol. Chem., 279(21):22118-22123 (2004)), which confirmed that mesotrypsin treatment enhances the invasive potential of the prostate cancer cells (FIG. 3C).

Whereas the WPE1 cell series models progression from premalignant lesion to invasive cancer, alternative cell lines were used to assess a possible role for mesotrypsin in the later stages of prostate cancer represented by metastasis and progression to androgen independence. The androgen independent prostate cancer cell line PC-3 and its derivatives show very high transcriptional levels of PRSS3 (Dozmorov et al., Prostate, 69(10):1077-1090 (2009)) (shown for the highly metastatic derivative PC3-M (Kozlowski et al., Cancer Res., 44(8):3522-3529 (1984)) in FIG. 2B). Both NB26 cells and PC3-M cells were used to probe the effects of endogenous levels of mesotrypsin expression on cellular invasion using RNA interference approaches (FIGS. 4A-D). Effective knockdown of mesotrypsin expression by stable transduction with a lentiviral shRNA construct targeting PRSS3 (FIGS. 4A and B) resulted in dramatically reduced invasion in Matrigel transwell invasion assays for both WPE1-NB26 cells (FIG. 4C) and PC3-M cells (FIG. 4D). In another experiment, PC3-M cells, in which PRSS3 expression was suppressed by shRNA knockdown, exhibited reduced metastatic potential in vivo in an orthotopic xenograft model of spontaneous metastasis (FIG. 9).

These results indicate that mesotrypsin is involved in prostate cancer invasion and metastasis, and indicates that inhibition of mesotrypsin can be used to treat cancers including prostate cancer. In addition, the results provided herein indicate that polypeptide inhibitors of serine proteases (e.g., BPTI-K15R/R17G) can be designed to have increased affinity and effective stability toward mesotrypsin, thereby providing polypeptide-based drugs for treating cancer. Such polypeptide-based drugs can exhibit high solubility, low toxicity, limited antigenicity, stability to digestion, and absorption into the bloodstream, and retention of activity after oral administration.

The results provided herein that indicate that mesotrypsin is highly upregulated and involved in invasion and metastasis of advanced, androgen-independent prostate cancer cells further suggests that mesotrypsin may offer a therapeutic target for androgen-independent prostate cancers.

### Example 3

### Identifying Mesotrypsin Substrates that May Lead to Malignant Morphology and Invasion

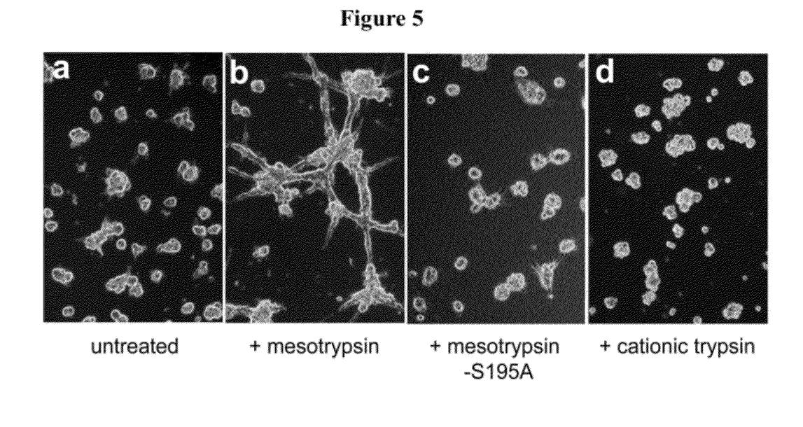

Treatment of prostate cancer cells of the WPE1 progression series with recombinant mesotrypsin stimulated malignant 3D growth morphology and Matrigel invasion (FIG. 3), while knockdown of mesotrypsin blocked invasion (FIG. 4). These effects were dependent upon the proteolytic activity of mesotrypsin, as the catalytically inactive mesotrypsin-S195A mutant exhibited no comparable effect (FIG. 5C), and the effects were highly specific to mesotrypsin, as they cannot be recapitulated by the closely homologous human cationic trypsin (FIG. 5D). These results indicate that mesotrypsin promotes malignant morphology by targeting unique substrates not susceptible to cleavage by other similar serine proteases. Toward known physiological protein substrates of other trypsins, mesotrypsin is a poor enzyme. It fails to activate pancreatic zymogens, and also shows reduced capacity to degrade trypsinogens (Szmola et al., J. Biol. Chem., 278(49):48580-48589 (2003)). Compared to other trypsins, mesotrypsin is also substantially compromised in its ability to cleave protease activated receptors (PARs) (Grishina et al., Br. J. Pharmacol., 146(7):990-999 (2005); Knecht et al., J. Biol. Chem., 282(36):26089-26100 (2007); and Wang et al., J. Neurochem., 99(3):759-769 (2006)). Despite minimal activity toward these classic trypsin substrates, mesotrypsin displays enhanced catalytic activity, relative to other trypsins, toward one class of protein substrates: the endogenous polypeptide protease inhibitors known as the ‘canonical’ inhibitors (Salameh et al., J. Biol. Chem., 285(3):1939-1949 (2010); Salameh et al., J. Biol. Chem., 283(7): 4115-4123 (2008); and Szmola et al., J. Biol. Chem., 278(49):48580-48589 (2003)). Based on the specificity of the effects observed in prostate cancer cells (FIG. 5), the endogenous canonical serine protease inhibitors were examined as possible substrates targeted for cleavage by mesotrypsin, through which mesotrypsin may promote prostate cancer malignancy. Using an affinity-based proteomic screen to identify endogenous trypsin inhibitors, secreted by prostate cancer cells, which are proteolyzed by mesotrypsin, the amyloid precursor protein (APP), which contains a canonical Kunitz protease inhibitor domain, was identified as a highly specific mesotrypsin substrate (Salameh et al., J. Biol. Chem., 285(3):1939-1949 (2010)). The site of cleavage was identified within the Kunitz domain, the kinetics of cleavage were characterized using HPLC and spectroscopic assays, and importantly, cleavage of APP by mesotrypsin was found to compromise its ability to inhibit both cationic trypsin and its primary physiological target Factor XIa. These results indicate that a possible mechanism by which mesotrypsin promotes cancer may involve proteolysis of secreted APP (and possibly other similar inhibitors) and ablation of protease inhibitory function. Mesotrypsin might contribute to the prothrombotic state of the tumor microenvironment, stimulating thrombin activation, signaling through PARs, platelet-tumor aggregation, adhesion to endothelium and subendothelial matrix, metastasis, tumor growth, and angiogenesis.

To screen additional endogenous canonical serine protease inhibitors produced by prostate cancer cells as candidate mesotrypsin substrates, a survey of the canonical protease inhibitors in the human genome was performed filtering to select only those inhibitors for which the P1 residues of one or more inhibitory domains are either Lys or Arg, thereby matching the primary specificity of mesotrypsin. This revealed 12 candidate substrates (Table 1). Transcriptional profiling of the cell lines in the WPE-1 prostate cancer progression series was performed using Affymetrix GeneChip Human Genome U133 Plus 2.0 microarrays. Relative transcription levels of the canonical inhibitor genes included in the microarray are indicated in Table 1.

TABLE 1
Human canonical inhibitors

| | | P₁ | Expression in |
|---|---|---|---|
| Inhibitor | Gene | residue | WPE-1 series |
| I1 - Kazal family | | | |
| SPINK1 | SPINK1 | K | very low |
| SPINK2 | SPINK2 | R | very low |
| SPINK6 | SPINK6 | R | very low |
| I2 - Kunitz-BPTI family | | | |
| HAI1 domains 1 & 2 | SPINT1 | K, R | uniformly high |
| HAI2 domains 1 & 2 | SPINT2 | R, R | uniformly high |
| amyloid precursor protein | APP | R | uniformly high |
| APLP2 | APLP2 | R | ND |
| WFIKKN1 | WFIKKN1 | R | very low |
| TFPI-1 domains 1 & 2 | TFPI | K, R | upreg. in |
| | | | progression |
| TFPI-2 domain 1 | TFPI2 | R | moderate |
| bikunin domain 2 | AMBP | R | ND |
| I17 - WAP-type family | | | |
| WFDC2 (HE4, WAP5) | WFDC2 | R | ND |

Expression levels in the WPE-1 cell progression series are validated initially for HAI1, HAI2, TFPI-1, TFPI-2, and the three canonical inhibitors not included in the microarrays, using qRT/PCR. For candidate transcripts expressed at significant levels in the prostate cells, Western blotting is used to assess expression at the protein level and then to evaluate sensitivity to mesotrypsin digestion. For APP and additional candidates found to be efficiently cleaved by mesotrypsin, the candidates are assessed for a role in the malignant phenotype using RNAi knockdown techniques.

### Example 4

### Signaling Mechanism by which Mesotrypsin Stimulates COX-2 Transcription

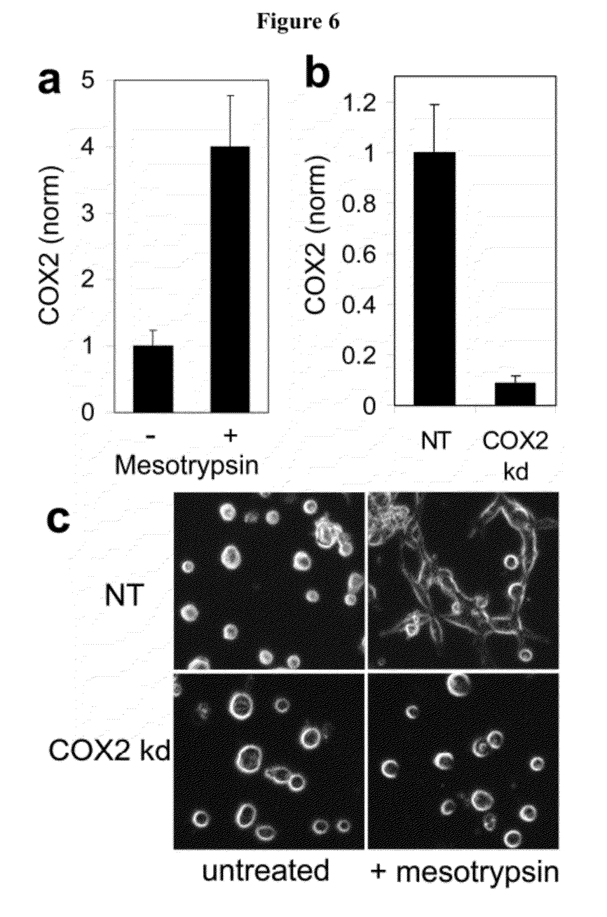

The striking changes in 3D phenotype and invasion induced by mesotrypsin suggest that a complex program of transcriptional changes may be initiated by mesotrypsin. To identify genes regulated by mesotrypsin, transcriptional profiling of the WPE1 cell series grown in 3D was performed, with and without recombinant mesotrypsin treatment, using Affymetrix GeneChip Human Genome U133 Plus 2.0 microarrays detecting 54,695 transcripts. One gene strikingly upregulated by mesotrypsin was COX-2. This result was validated for WPE-1 NB11 cells by qRT/PCR (FIG. 6A). As COX-2 is often upregulated with cancer progression and has been implicated in prostate cancer cell survival, invasion, and progression, it was hypothesized that upregulation of COX-2 might be a pathway by which mesotrypsin promotes malignant progression. To test this hypothesis, COX-2 expression was suppressed in NB11 cells by stable transduction with a lentiviral shRNA construct (FIG. 6B). After culturing the stably transduced cells in 3D with or without recombinant mesotrypsin, it was found that the malignant morphology promoted by mesotrypsin was dependent upon COX-2, as knockdown of COX-2 completely suppressed morphological changes in response to mesotrypsin (FIG. 6C).

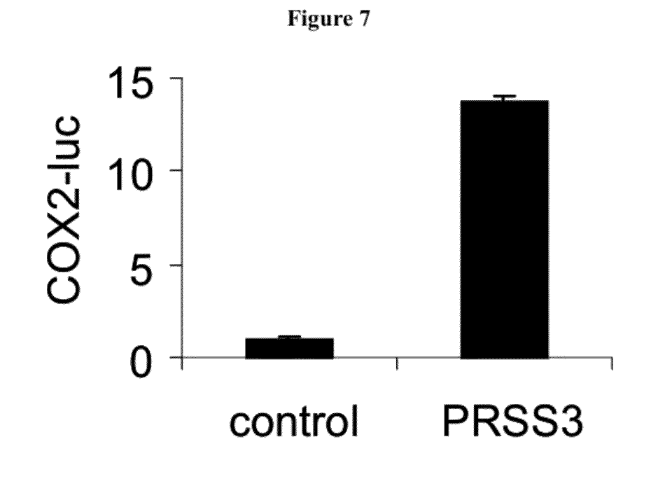

Several consensus sequences in the COX-2 promoter, including those for NF-κB, NF-IL6, ATF/CRE, and an E-box, have been identified by others as cis-acting regulatory sequences involved in COX-2 induction in response to a variety of stimuli in different species and cell types. A construct featuring a 724 nucleotide promoter sequence of COX-2 fused to a luciferase reporter was obtained along with a series of mutant constructs in which specific promoter elements have been altered. Mesotrypsin treatment of NB11 cells transfected with the wild-type reporter construct and grown in 3D substantially increased expression of the luciferase reporter (FIG. 7).

### Example 5

### Role of Mesotrypsin in Promoting Tumor Growth, Invasion, and Spontaneous Metastasis in Mouse Orthotopic Xenograft Models of Prostate Cancer Progression

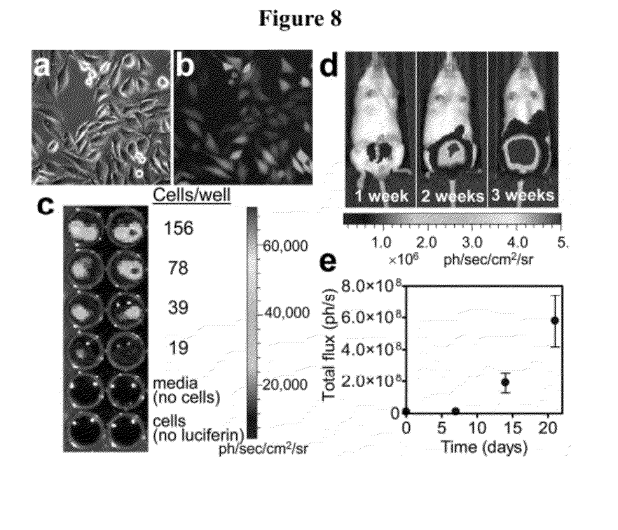

As indicated herein, PC3-M cells express 22-fold higher levels of mesotrypsin than RWPE-1 normal prostate epithelial cells, and 3.5-fold higher levels of mesotrypsin than WPE1 NB26 cells (FIG. 2B), making this an appropriate cell line for assessing the effect on metastasis of very high endogenous levels of mesotrypsin. The metastatic potential of PC3-M cells transduced with a lentiviral shRNA construct designed to suppress mesotrypsin expression (FIG. 4B) was compared to the metastatic potential of PC3-M cell transduced with a nontarget control virus. Stably-transduced, puromycin-selected cells were superinfected with the lentiviral pSIN-luc construct for firefly luciferase to visualize tumor growth and metastasis by BLI. Near 100% transduction efficiency was achieved with this construct in these cells (FIGS. 8A and 8B), and luciferase activity was detected from fewer than 20 cells (FIG. 8C). PC3-M cells implanted orthotopically form rapidly growing tumors (FIGS. 8D and 8E) characterized by widespread metastasis. Metastases appeared first in the lungs: by 9 days post-implantation, 40% of mice exhibited metastases detectable by ex vivo BLI at necropsy, and by 14 days, more than 90% of mice exhibited pulmonary metastases. In mice sacrificed at 3 weeks post-implantation, additional metastases were detected in the liver, kidney, spleen, and diaphragm. Lung and liver are common sites of prostate cancer metastases in humans (Shah et al., Cancer Res., 64(24):9209-9216 (2004)), although this mouse model did not recapitulate metastasis to bone, the most frequent site of human metastasis. PRSS3 knockdown attenuated tumor growth (FIG. 9A). A larger effect was observed on meta-stasis, with far lower tumor burden present in the lungs of animals that received PRSS3-knockdown cells compared with controls (FIG. 9B-D). Metastatic tumor burden was assessed at necropsy by ex vivo BLI; staining lung specimens for human cytokeratins (FIG. 9E) and H&E (FIG. 9F) confirmed that bioluminescent signal was corroborated by further evidence of metastasis.

### Example 6

### Determinants of Affinity and Proteolytic Stability in Interactions of Kunitz Family Protease Inhibitors with Mesotrypsin

### Production of Recombinant Proteins

Recombinant human mesotrypsinogen and a catalytically inactive S195A mutant were expressed in E. coli, isolated from inclusion bodies, refolded, purified, and activated with bovine enteropeptidase as described elsewhere (Salameh et al., J. Boil. Chem., 283: 4115-4123 (2008)). Kunitz domain inhibitors were expressed in the methlotrophic yeast Pichia pastoris under control of the alcohol oxidase (AOX1) promoter using the expression vector pPICZαA (Invitrogen); construction of the APPI-wt, BPTI-wt, and P₁ mutant expression constructs is described elsewhere (Navaneetham et al., J. Biol. Chem., 280:36165-36175 (2005) and Navaneetham et al., (Jul. 20, 2010) J. Biochem., 10.1093/jb/mvq080). Additional mutations were introduced using the QuikChange kit (Stratagene) according to manufacturer protocols; mutant plasmids were verified by sequencing. Expression constructs were linearized with Sad (New England BioLabs) and transformed via electroporation into P. pastoris X33 (Invitrogen). Chromosomal integration was verified by PCR amplification of genomic DNA using AOX1 forward and reverse primers. A number of verified transformants were screened for expression in small scale liquid cultures; those with media revealing the highest levels of trypsin inhibitory activity were selected for large (1 L) scale expression and purification. Expression cultures grown in BMMY medium (buffered medium containing methanol and yeast nitrogen base) at 30° C. were harvested after 48 hours. The supernatant was subjected to ammonium sulfate precipitation (95% saturation) at room temperature. Protein pellets were resuspended in 20 mM Tris buffer, pH 7.8, and dialyzed overnight against 10 mM Tris buffer pH 7.8 using 3.5 kDa cutoff dialysis tubing (ThermoScientific). Dialyzed APPI samples were chromatographed on Q-sepharose FF (GE Healthcare) and eluted with a gradient of 0 to 100% buffer B (50 mM Tris pH 7.8, 1 M NaCl). Dialyzed BPTI samples were chromatographed on SP-sepharose FF (GE Healthcare) and eluted with a gradient of 0 to 100% buffer B (50 mM Tris pH 7.8, 1 M NaCl). Both APPI and BPTI proteins were further purified on a trypsin affinity column as described elsewhere (Salameh et al., J. Biol. Chem., 285:1939-1949 (2010)), and eluted with a gradient of 0-100% buffer B (150 mM HCl). Protein purity was verified by HPLC using a reversed phase Jupiter 4μ 90 Å C₁₂ column (Phenomenex), to ensure that each inhibitor chromatographed as a single peak. BPTI-R17M and BPTI-K15R/R17M were further purified by HPLC using a reversed phase C₁₈ column (Luna 250×10 mm 5μ) (Phenomenex) and a gradient of 0 to 100% acetonitrile in 0.1% TFA.

### Competitive Inhibition Studies

Mesotrypsin concentration was quantified by active-site titration using 4-nitrophenyl 4-guanidinobenzoate (Sigma) (Chase and Shaw, Biochem. Biophys. Res. Commun., 29:508-514 (1967)). Concentrations of APPI and BPTI variants were determined by titration with bovine trypsin (Sigma) as described elsewhere (Salameh et al., J. Biol. Chem., 283: 4115-4123 (2008)). Concentrations of the chromogenic substrate carboxybenzyl-Gly-Pro-Arg-p-nitroanalide (Z-GPR-pNA; Sigma) were determined by end point assay. Working stocks of enzyme, substrate, and inhibitors were prepared and assays conducted as described elsewhere (Salameh et al., J. Biol. Chem., 283: 4115-4123 (2008)) and Salameh et al., J. Biol. Chem., 285:1939-1949 (2010)). Briefly, reactions carried out at 37° C. in a Varian Cary-100 spectrophotometer were followed spectroscopically for 3-5 minutes, and initial rates were determined from the absorbance increase caused by the release of p-nitroaniline (ε₄₁₀=8480 M⁻¹ cm⁻¹) (DelMar et al., Anal. Biochem. 99:316-320 (1979)). Data were globally fitted by multiple regression to equation 1, the classic competitive inhibition equation, using Prism (GraphPad Software, San Diego Calif.). Reported inhibition constants are average values obtained from 2-4 independent experiments.

$$v = \frac{{{k}_{cat} [ E ]}_{0} [ S ]}{{K}_{m} ( 1 + [ I ] / {K}_{i} ) + [ S ]} \qquad ( 1 )$$

### Inhibitor Hydrolysis Studies

The depletion of intact APPI and BPTI variants in time course incubations with active mesotrypsin was monitored by HPLC and 16% SDS-Tricine PAGE (Schagger and von Jagow, Anal. Biochem., 166:368-379 (1987) and Gallagher, (2001) in Current protocols in molecular biology (Ausubel, F. M. ed.), pp 10.10.10-10.11.34, J. Wiley, New York). Incubations of mesotrypsin with BPTI mutants were carried out in 0.1 M Tris-HCl, pH 8.0, and 1 mM CaCl₂ at 37° C.; BPTI concentration was 50 μM and mesotrypsin concentration was 2.5-5 μM. Aliquots for HPLC analysis were withdrawn at periodic intervals, adjusted to 6 M urea and 2 mM DTT, incubated for 10 minutes at 37° C., quenched by acidification to pH 1 and then frozen at −20° C. until analyzed as described elsewhere (Salameh et al., J. Biol. Chem., 283: 4115-4123 (2008)). Incubations of mesotrypsin with APPI mutants were carried out similarly, except that APPI concentration was 50 μM and mesotrypsin concentration was 500 nM (for APPI-R15K and APPI-R15K/M17R) or 50 nM (for APPI-M17R). APPI hydrolysis time point samples were not denatured or reduced; instead samples were quenched immediately by acidification to pH 1 and then frozen at −20° C. until analyzed. Enzyme, Kunitz inhibitors, and hydrolysis products were resolved on a 50×2.0 mm Jupiter 4μ90 Å C₁₂ column (Phenomenex) with a gradient of 0 to 100% acetonitrile in 0.1% TFA at a flow rate of 0.6 ml/min over 50 minutes. Peak integration to quantify the disappearance of intact Kunitz inhibitors over time was carried out as described elsewhere (Salameh et al., J. Biol. Chem., 283: 4115-4123 (2008)); initial rates were obtained by linear regression using a minimum of 6 data points within the initial linear phase of the reaction, and not exceeding 25% conversion of intact inhibitor to hydrolysis products. Hydrolysis rates reported for each inhibitor represent the average of 2-4 independent experiments.

### Crystallization of Mesotrypsin-APPI Complexes

Complexes of catalytically inactive mesotrypsin-S195A with APPI and APPI-R15K were crystallized by vapor diffusion. Mesotrypsin-S195A dissolved in 1 mM HCl and APPI dissolved in 10 mM NaOAc pH 6.5 were mixed in a 1:1 stoichiometric molar ratio, to achieve a total protein concentration of 3-6 mg/mL. Crystals were grown at 22° C. in hanging drops, over a reservoir of 4.5 M Na-formate. Drops (4 μL) were prepared by mixing equal volumes of protein and reservoir solutions. Crystals (0.1×0.2×0.1 mm) appeared within 4 days and grew over the course of three weeks. Crystals were harvested, soaked in a cryoprotectant solution (4.5 M Na-formate and 17.5% glycerol) and flash-frozen in liquid N₂.

### X-Ray Data Collection, Structure Solution and Model Refinement

Synchrotron X-ray data were collected from crystals at 100 K using an ADSC Quantum-4 CCD detector at beam line X12-B, National Synchrotron Light Source, Brookhaven National Laboratory. Mesotrypsin/APPI and mesotrypsin/APPI-R15K complex crystals belong to space group P22₁2₁ with unit cell parameters of a=92.79, b=130.067, c=132.33, and a=92.93, b=131.35, c=131.9, respectively, and α=β=γ=90°. Diffraction data were measured to 2.48 and 2.38-Å resolution for mesotrypsin/APPI and mesotrypsin/APPI-R15K complexes, respectively. The automation package ELVES (Holton and Alber, Proc. Natl. Acad. Sci. USA., 101:1537-1542 (2004)) was used to direct MOLFLM (Leslie, (1992) Joint CCP4+ESF-EAMCB Newsletter on Protein Crystallography 26) for indexing and integration, and SCALA for scaling and merging the reflections (Acta. Crystallogr. D Biol. Crystallogr., 50:760-763 (1994)). The mesotrypsin/APPI structure was solved by molecular replacement using the program Phaser (McCoy et al., Acta. Crystallogr. D Biol. Crystallogr., 61:458-464 (2005)) operated by PHENIX (Adams et al., Acta. Crystallogr. D Biol. Crystallogr., 58:1948-1954 (2002)) using mesotrypsin and APPI structures as pieces of a complete search model, allowing independent translation and rotation of each piece. The search model was derived from previous structures of mesotrypsin (PDB ID 2R9P, chain A) (Salameh et al., J. Biol. Chem., 283: 4115-4123 (2008)) and APPI (PDB ID 1ZJD, chain B) (Navaneetham et al., J. Biol. Chem., 280:36165-36175 (2005)). The solved structure of the mesotrypsin/APPI complex was used as a model for the mesotrypsin/APPI-R15K complex structure. The successful solution contained 4 copies of each protein in the asymmetric unit, forming 4 canonical trypsin-APPI complexes. Cycles of manual rebuilding in COOT (Emsley and Cowtan, Acta. Crystallogr. D Biol. Crystallogr., 60:2126-2132 (2004)) were alternated with automated refinement using the refinement module of the PHENIX software suite (Afonine et al., CCP4 Newsletter 42 (2005)). A test set comprised of 10% of the total reflections was excluded from refinement to allow calculation of the free R factor. TLS refinement was employed, with each protein chain assigned to a separate TLS group. Waters, ions, and alternative conformations of protein residues were added using COOT (Emsley and Cowtan, Acta. Crystallogr. D Biol. Crystallogr., 60:2126-2132 (2004)). All superpositions and structure figures were created using the graphics software PyMol (http://pymol.sourceforge.net) (DeLano, The PyMOL Molecular Graphics System. DeLano Scientific, San Carlos, Calif., USA (2002)).

### Abbreviations

BPTI, bovine pancreatic trypsin inhibitor; APPI, Alzheimer's amyloid-protein precursor inhibitor; Z-GPR-pNA, Carboxybenzyl-Gly-Pro-Arg-p-nitroanalide; βME, 2-mercaptoethanol; TFA, trifluoroacetic acid; SDS-PAGE, sodium dodecyl sulfate-polyacrylamide gel electrophoresis; HPLC, high-pressure liquid chromatography; BSA, bovine serum albumin; DTT, DL-dithiothreitol; DMSO, dimethyl sulfoxide; PCR, polymerase chain reaction. Substrate residues surrounding the cleavage site are designated by the nomenclature of Schechter and Berger (Schechter and Berger, Biochem. Biophys. Res. Commun., 27:157-162 (1967): starting from the scissile bond, substrate residues are numbered P₁, P₂, P₃, etc. in the direction of the N terminus (collectively the non-primed residues), and P₁′, P₂′, P₃′, etc. in the direction of the C terminus (collectively the primed residues). Corresponding enzyme subsites are numbered S₁, S₂, S₃, etc.

### Results

### BPTI and APPI Show Striking Differences in Binding and Susceptibility to Cleavage by Mesotrypsin

APPI bound to mesotrypsin 100× more tightly than BPTI, and was cleaved by mesotrypsin >300× more rapidly than BPTI (Salameh et al., J. Biol. Chem., 283: 4115-4123 (2008) and Salameh et al., J. Biol. Chem., 285:1939-1949 (2010)). To identify sequence or structural differences between these inhibitors potentially responsible for the differences in their interactions with mesotrypsin, sequence and structural alignments were performed (FIG. 10). BPTI and APPI share 44% sequence identity (FIG. 10A) and a highly conserved three-dimensional structure (FIG. 10B). The conventional BPTI numbering system was used for designating analogous residues of APPI. Focusing on the canonical loop, which makes the majority of the inhibitor-enzyme close contacts, two non-identical residues were identified: the P₁ position (Lys-15 in BPTI, Arg-15 in APPI) and the P₂′ position (Arg-17 in BPTI, Met-17 in APPI). To dissect the roles of the P₁ residue, the P₂′ residue, and the BPTI vs. APPI scaffold in accounting for the striking differences in mesotrypsin binding affinities and cleavage rates, a series of single and double mutants were generated which interchanged P₁ and P₂′ residues, creating variants in which inhibitors differ only at the P₁ or the P₂′ position, or have identical canonical loops in the context of different scaffolds.

As a measure of the binding affinity of mesotrypsin for BPTI and APPI variants, equilibrium inhibition constants (Kᵢ) were obtained using classic competitive inhibition experiments in which cleavage of the chromogenic substrate Z-GPR-pNA by mesotrypsin in the presence of varying concentrations of inhibitor was monitored. All of the Kunitz variants tested exhibited kinetics of inhibition that were well described by a strictly competitive model, as reported elsewhere for the inhibition of mesotrypsin by BPTI-wt (Salameh et al., J. Biol. Chem., 283: 4115-4123 (2008)) and APPI-wt (Salameh et al., J. Biol. Chem., 285:1939-1949 (2010)). Values of Kᵢ for all of the Kunitz variants are summarized in Table 2. The competitive inhibition equation used to determine Kᵢ also described the enzymatic reaction of one substrate in the presence of an alternative, competing substrate, with the added condition that the observed Kᵢ for the alternative substrate must be equivalent to its Michaelis constant Kₘ (Cornish-Bowden, (1995) Fundamentals of enzyme kinetics, pp 105-108, Portland Press, London). Thus, mesotrypsin cleaves both BPTI and APPI at the reactive site peptide bond (Salameh et al., J. Biol. Chem., 283: 4115-4123 (2008) and Salameh et al., J. Biol. Chem., 285:1939-1949 (2010). The measured Kᵢ values also represented Kₘ values for cleavage of the Kunitz inhibitors as substrates of mesotrypsin.

TABLE 2
Kinetic constants of mesotrypsin with APPI and BPTI variants

| Inhibitor | P₁ | P₂′ | k_{cat} (s⁻¹) | Kᵢ (M) | k_{cat}/Kₘ (s⁻¹M⁻¹) |
|---|---|---|---|---|---|
| APPI-wt | R | M | 4.32 ± 0.40 × 10⁻² | 1.36 ± 0.19 × 10⁻⁷ | 3.18 ± 0.53 × 10⁵ |
| APPI-R15K | K | M | 3.21 ± 0.60 × 10⁻³ | 3.50 ± 0.14 × 10⁻⁷ | 9.2 ± 1.7 × 10³ |
| APPI-M17R | R | R | 5.36 ± 0.16 × 10⁻² | 9.20 ± 0.99 × 10⁻⁷ | 5.83 ± 0.65 × 10⁴ |
| APPI-R15K/M17R | K | R | 3.37 ± 0.63 × 10⁻³ | 1.53 ± 0.01 × 10⁻⁶ | 2.20 ± 0.41 × 10³ |
| BPTI-wt | K | R | 1.85 ± 0.23 × 10⁻⁴ | 1.35 ± 0.19 × 10⁻⁵ | 1.37 ± 0.23 × 10¹ |
| BPTI-K15R | R | R | 8.7 ± 3.4 × 10⁻⁴ | 2.38 ± 0.20 × 10⁻⁶ | 3.6 ± 1.5 × 10² |
| BPTI-R17M | K | M | 1.06 ± 0.06 × 10⁻⁴ | 7.61 ± 0.54 × 10⁻⁷ | 1.39 ± 0.13 × 10² |
| BPTI-K15R/R17M | R | M | 5.14 ± 0.23 × 10⁻⁴ | 2.18 ± 0.01 × 10⁻⁷ | 2.36 ± 0.11 × 10³ |

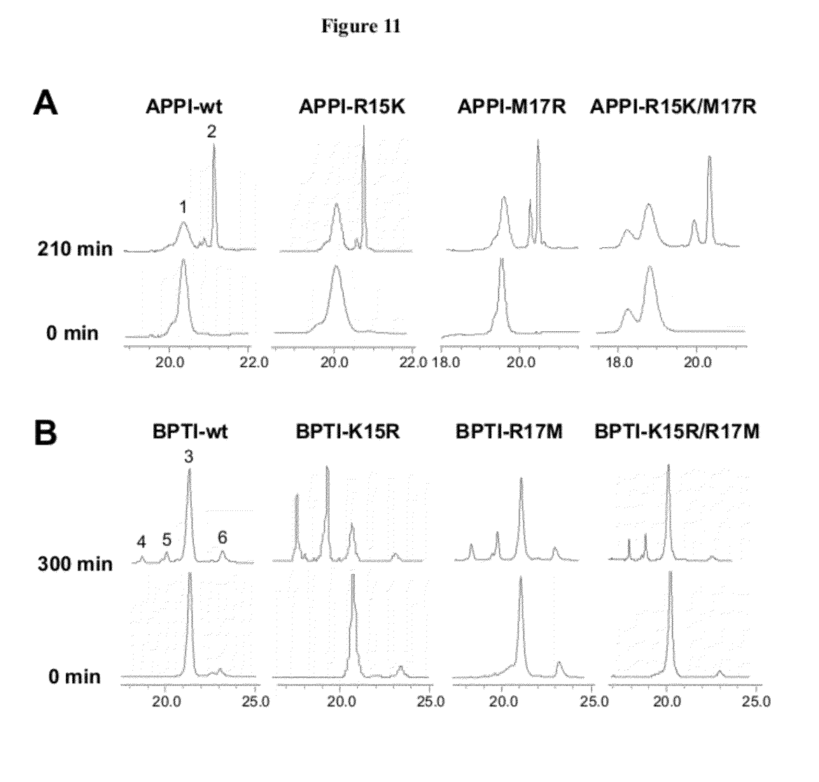

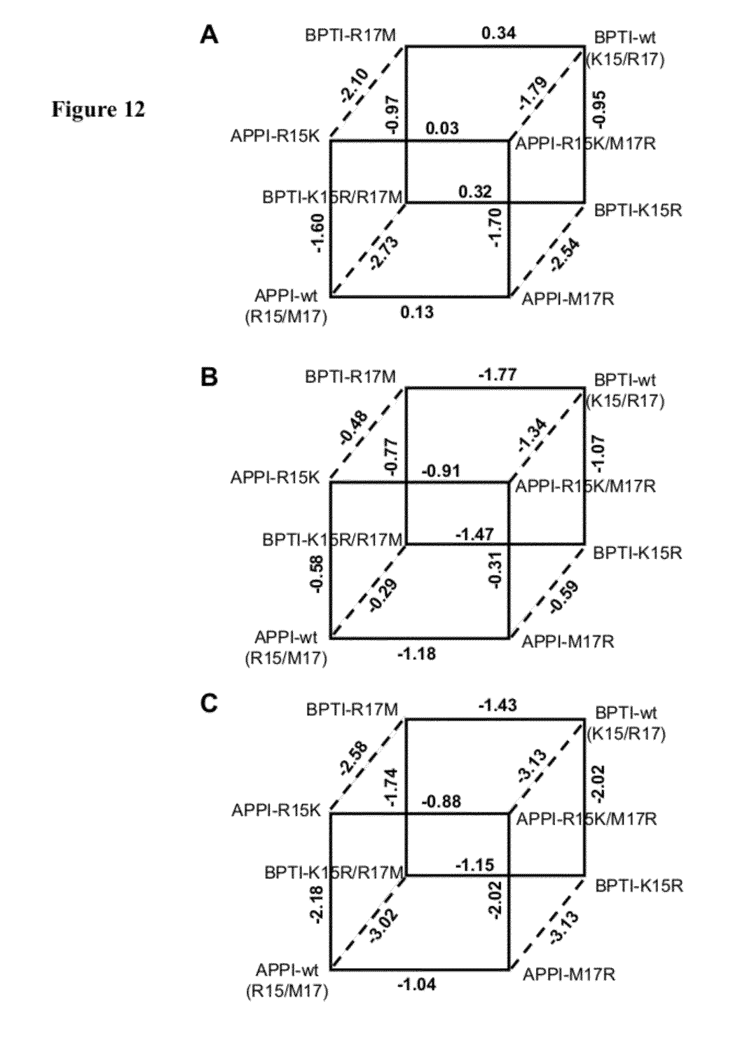

SDS-PAGE and HPLC assays were used to monitor inhibitor cleavage in time course incubations with mesotrypsin and to calculate rates of catalysis (k_{cat}). HPLC peaks corresponding to BPTI and APPI cleavage products were clearly resolved from starting materials (FIG. 11), and peak integration enabled quantification of cleavage rates. Because hydrolysis studies were performed using Kunitz inhibitor concentrations that were more than 10-fold higher than the Kₘ values, the enzyme binding capacity was saturated, and the measured rates of hydrolysis approximate true catalytic rate constants (k_{cat}). Values of k_{cat} for all of the Kunitz variants are summarized in Table 2. The k_{cat} value reported herein for BPTI-wt was about 40% higher than that which was reported elsewhere (Salameh et al., J. Biol. Chem., 283: 4115-4123 (2008)); this updated value reflects the average of multiple additional experiments.

### The Effect of P₁ Substitution on Binding Affinity and Hydrolysis

To dissect the contribution of the P₁ residue in BPTI and APPI interactions with mesotrypsin, the kinetic data in Table 2 were used to compare variants that differ in possessing Arg vs. Lys at P₁, but are otherwise identical (Table 3). Among the four pairs of possible comparisons, it was apparent that Arg at P₁ (vs. Lys) favors both tighter binding (by a factor of 2-6) and more rapid cleavage (by a factor of 5-16). Using measured Kᵢ values to represent Kₘ (see above), the specificity constant k_{cat}/Kₘ was calculated for each inhibitor considered as a substrate of mesotrypsin. Arg at P₁ (vs. Lys) resulted in a 17-35-fold enhancement of k_{cat}/Kₘ (Table 3). These data demonstrate that mesotrypsin exhibits a moderate preference for binding of inhibitors with Arg vs. Lys at the P₁ position, and a more pronounced preference for cleavage of substrates with Arg vs. Lys at the P₁ position.

TABLE 3
Impact of Arg vs. Lys at the P₁ position on mesotrypsin hydrolysis (k_{cat}),
association equilibrium constant (Kₐ = 1/Kᵢ), and substrate specificity (k_{cat}/Kₘ)

| | | | | k_{cat} | | 1/Kᵢ | | k_{cat}/Kₘ | |
|---|---|---|---|---|---|---|---|---|---|
| numerator | denominator | | | fold | | fold | | fold | |
| (P₁ = R) | (P₁ = K) | scaffold | P₂′ | diff | ΔΔG_{cat} | diff | ΔΔGₐ° | diff | ΔΔG_{T}^{‡} |
| APPI-wt | APPI-R15K | APPI | M | 13.5 | −1.60 | 2.6 | −0.58 | 34.6 | −2.18 |
| BPTI-K15R | BPTI-wt | BPTI | R | 4.7 | −0.95 | 5.7 | −1.07 | 26.5 | −2.02 |
| APPI-M17R | APPI- | APPI | R | 15.9 | −1.70 | 1.7 | −0.31 | 26.5 | −2.02 |
| | R15K/M17R | | | | | | | | |
| BPTI- | BPTI-R17M | BPTI | M | 4.8 | −0.97 | 3.5 | −0.77 | 16.9 | −1.74 |
| K15R/R17M | | | | | | | | | |

### The Effect of P₂′ Substitution on Binding Affinity and Hydrolysis

Mesotrypsin possesses an Arg residue at position 193, where nearly all serine proteases including other trypsins have a highly conserved Gly, and Arg-193 has been found to be a major determinant of mesotrypsin's atypical catalytic properties (Szmola et al., J. Biol. Chem., 278:48580-48589 (2003) and Katona et al., J. Mol. Biol., 315:1209-1218 (2002)). Mesotrypsin Arg-193 undergoes conformational change upon binding to BPTI in order to ameliorate steric conflict between Arg-193 and BPTI Arg-17, the P₂′ residue (Salameh et al., J. Biol. Chem., 283: 4115-4123 (2008)). Because of the close contact between mesotrypsin Arg-193 and the inhibitor P₂′ residue, the identity of the P₂′ residue, in BPTI a basic charged Arg and in APPI a more hydrophobic Met, might be a factor influencing both binding affinity and inhibitor cleavage rates. By comparing kinetic data extracted from Table 2 for the four pairs of Kunitz inhibitors that differ only at the P₂′ residue (Table 4), Met (vs. Arg) at the P₂′ position was found to result in stronger binding by a factor of 4-18. Surprisingly, the effects on rates of catalysis were much more minimal; no significant effect for P₂′ Met vs. Arg was found in the APPI variants, and only a slight effect on BPTI cleavage rate, where Met (vs. Arg) slowed k_{cat} by ˜2-fold, was found (Table 4). The combined contributions from k_{cat} and Kₘ resulted in a 4-10-fold difference in substrate specificity constant k_{cat}/Kₘ, favoring the Kunitz variants with Met at the P₂′ position.

TABLE 4
Impact of Met vs. Arg at the P₂′ position on mesotrypsin hydrolysis (k_{cat}),
association equilibrium constant (Kₐ = 1/Kᵢ), and substrate specificity (k_{cat}/Kₘ)

| | | | | k_{cat} | | 1/Kᵢ | | k_{cat}/Kₘ | |
|---|---|---|---|---|---|---|---|---|---|
| numerator | denominator | | | fold | | fold | | fold | |
| (P₂′ = M) | (P₂′ = R) | scaffold | P₁ | diff | ΔΔG_{cat} | diff | ΔΔGₐ° | diff | ΔΔG_{T}^{‡} |
| APPI-wt | APPI-M17R | APPI | R | 0.8 | 0.13 | 6.8 | −1.18 | 5.5 | −1.04 |
| BPTI-R17M | BPTI-wt | BPTI | K | 0.6 | 0.34 | 17.7 | −1.77 | 10.2 | −1.43 |
| APPI-R15K | APPI- | APPI | K | 1.0 | 0.03 | 4.4 | −0.91 | 4.2 | −0.88 |
| | R15K/M17R | | | | | | | | |
| BPTI- | BPTI-K15R | BPTI | R | 0.6 | 0.32 | 11.0 | −1.47 | 6.5 | −1.15 |
| K15R/R17M | | | | | | | | | |

### The Inhibitor Scaffold is a Major Determinant of Inhibitor Susceptibility to Hydrolysis

A surprising outcome from the kinetic analyses of the Kunitz inhibitors came from examining the contributions of the scaffolds of BPTI and APPI to differences in inhibitor binding and cleavage. Pairwise comparisons of BPTI and APPI variants sharing identical sequence throughout the canonical binding loop allowed the role of the protein scaffolds to be assessed, defined as the protein structure exclusive of the P₅-P₃′ binding loop (Table 5). The APPI scaffold was found to confer minimal (2-fold) enhanced affinity, but an impressive 18-84-fold enhancement in k_{cat} relative to the BPTI scaffold (Table 5). The aggregate contributions from k_{cat} and Kₘ resulted in substrate specificity constants k_{cat}/Kₘ more than two orders of magnitude greater for the Kunitz inhibitors bearing the APPI scaffold, compared to the sequence-matched BPTI variants (Table 5). Thus, the enhanced vulnerability of APPI to mesotrypsin hydrolysis (relative to BPTI) was attributable not only to the canonical binding loop sequence, but was largely determined by elements of the scaffold, despite the facts that the cleavage site was embedded within the canonical binding loop, and that this loop makes the majority of close contacts with the enzyme.

TABLE 5
Impact of APPI vs. BPTI scaffold on mesotrypsin hydrolysis (k_{cat}),
association equilibrium constant (Kₐ = 1/Kᵢ), and substrate specificity (k_{cat}/Kₘ).

| | | | | k_{cat} | | 1/Kᵢ | | k_{cat}/Kₘ | |
|---|---|---|---|---|---|---|---|---|---|
| numerator | denominator | | | fold | | fold | | fold | |
| (APPI) | (BPTI) | P₁ | P₂′ | diff | ΔΔG_{cat} | diff | ΔΔGₐ° | diff | ΔΔG_{T}^{‡} |
| APPI-M17R | BPTI-K15R | R | R | 61.9 | −2.54 | 2.6 | −0.59 | 160.3 | −3.13 |
| APPI-R15K | BPTI-R17M | K | M | 30.3 | −2.10 | 2.2 | −0.48 | 65.8 | −2.58 |
| APPI- | BPTI-wt | K | R | 18.2 | −1.79 | 8.8 | −1.34 | 160.5 | −3.13 |
| R15K/M17R | | | | | | | | | |
| APPI-wt | BPTI- | R | M | 84.1 | −2.73 | 1.6 | −0.29 | 134.5 | −3.02 |
| | K15R/R17M | | | | | | | | |

Structural Insights into Differential Proteolytic Stability of BPTI and APPI

The crystal structure of mesotrypsin bound to BPTI (PDB ID 2R9P) is provided elsewhere (Salameh et al., J. Biol. Chem., 285:1939-1949 (2010)), and two new crystal structures of mesotrypsin bound to APPI are provided herein. Comparisons of these structures may offer insights into the observed differences in inhibitor binding and hydrolysis. Because it is anticipated that APPI would be rapidly proteolyzed under crystallization conditions with active mesotrypsin, an essentially isostructural but inactive mutant of mesotrypsin featuring a Ser-195 to Ala mutation in the active site was used. Cocrystals of mesotrypsin-S195A were grown with both APPI-wt and APPI-R15K variants. The structures were solved by molecular replacement and refined against data extending to 2.4-2.5 Å resolution; Table 6 summarizes the crystal, data collection, and refinement statistics. Both structures contained four highly similar complexes in the asymmetric unit, each featuring a molecule of APPI bound in the canonical fashion at the mesotrypsin active site.

TABLE 6
Data collection and refinement statistics
for mesotrypsin/APPI complexes.

| | Mesotrypsin/APPI-wt | Mesotrypsin/APPI-R15K |
|---|---|---|
| PDB ID | 3L33 | 3L3T |
| Complexes per ASU | 4 | 4 |
| Space group | P22₁2₁ | P22₁2₁ |
| Unit cell, Å | 92.80, 130.06, 132.34, | 92.93, 131.35, 131.9, |
| | 90°, 90°, 90° | 90°, 90°, 90° |
| Resolution range, Å | 23.7-2.48 | 24.48-2.40 |
| Unique reflections | 56,879 | 63,638 |
| Completeness, % | 99.3 (95.8) | 99.8 (99.1) |
| Multiplicity | 14.2 (12.4) | 13.2 (8.6) |
| I/S.D. | 11.1 (1.5) | 13.3 (1.6) |
| R_{sym} | 0.26 (2.3) | 0.162 (1.27) |
| R_{cryst}/R_{free} | 19.73/25.53 | 18.33/23.69 |
| R.m.s.d. bonds, Å | 0.010 | 0.006 |
| R.m.s.d. angles, ° | 0.996 | 0.698 |

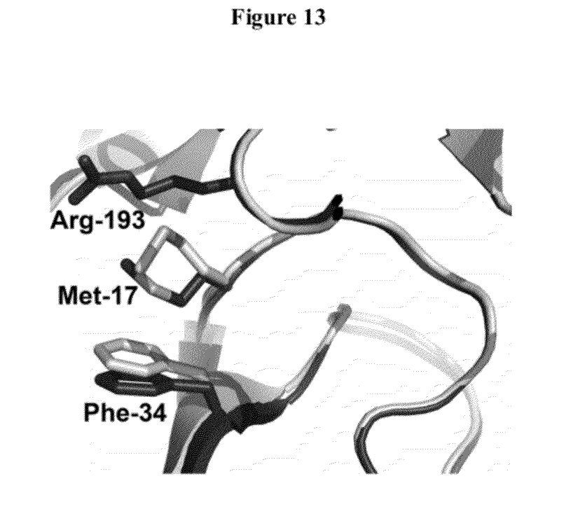

The overall fold of APPI and mode of interaction with mesotrypsin are similar to those found in structures of APPI complexed with bovine trypsin (PDB ID 1TAW) (Scheidig et al., Protein Sci., 6:1806-1824 (1997)) and rat anionic trypsin (PDB ID 1BRC) (Perona et al., J. Mol. Biol., 230:919-933 (1993)). The major difference between the mesotrypsin-APPI complex and these other trypsin-APPI complexes occurs in the vicinity of mesotrypsin Arg-193, where similarly to findings described elsewhere for the mesotrypsin-BPTI structure (Salameh et al., J. Biol. Chem., 283: 4115-4123 (2008)), the presence of Arg-193 forces a displacement of the P₂′ residue to ameliorate a steric clash. Comparing the mesotrypsin/APPI structure with the structure of bovine trypsin complexed with APPI (PDB ID 1TAW) (Scheidig et al., Protein Sci., 6:1806-1824 (1997)), the S_{δ} atom of Met-17 was displaced downward by Arg-193 by about 2.5 Å, in turn displacing Phe-34 downward by 1.5 Å (FIG. 13).

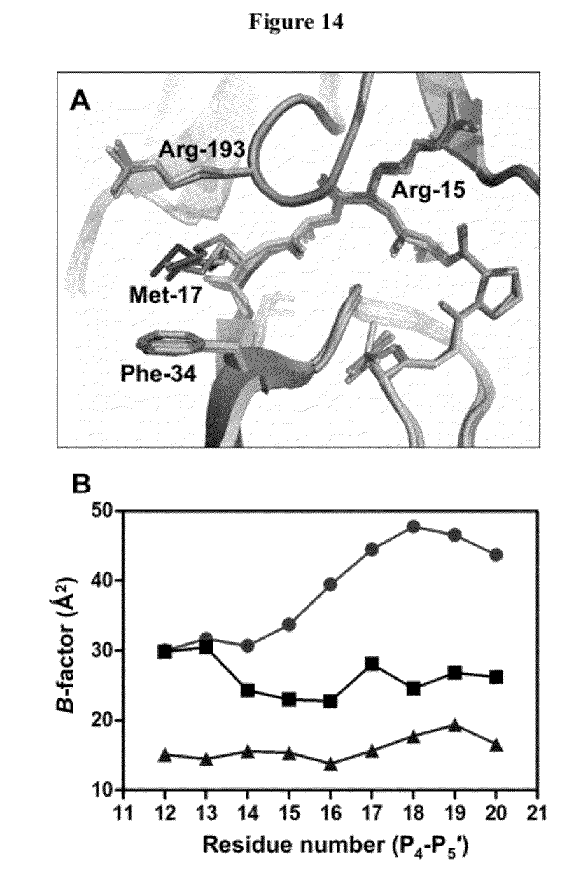

Several aspects of the data hint at an increased degree of mobility or flexibility of the primed side of the APPI canonical loop in complex with mesotrypsin, by comparison with BPTI in complex with mesotrypsin, and also by comparison with APPI bound to other trypsins. While electron density for the Met-17 side chain was not seen, it is somewhat diffuse, and conformations in the refined model vary slightly among the different copies of the molecule in the asymmetric unit (FIG. 14A). This contrasts with the mesotrypsin-BPTI structure (PDB ID 2R9P), in which the Arg-17 P₂′ residue was more precisely oriented, forming a water-bridged H-bond with Arg-193 of mesotrypsin (See FIG. 3B of Salameh et al., J. Biol. Chem., 283: 4115-4123 (2008)).

Another indication of the dynamic properties of APPI was found in analysis of B-factors, also referred to as crystallographic temperature factors, which reflect the fluctuations of atoms about their average positions and provide qualitative information about the residual mobility of regions in a crystal structure. The diffraction data were collected at 100 K where motions were considerably reduced, but flash cooling may generate a frozen static picture of protein dynamics under ambient conditions (Baum et al., Mol. Biol., 397:1042-1054 (2010)). The four copies of the mesotrypsin/APPI complex in the crystal differed considerably in their average B-factors, with the chain A/chain E complex possessing the lowest B-factors while the chain D/chain H complex possessed much higher B-factors throughout; however, the trends described here and plotted for the chain A/chainE complex in FIG. 14B were consistently observed across all complexes. The non-primed side residues of the canonical loop showed B-factors among the lowest in the molecule, indicative of a fairly rigid structure at the enzyme-inhibitor interface, as is typical in structures of serine proteases inhibited by canonical inhibitors. By contrast, the backbone atoms of the primed side residues of the APPI canonical loop showed significantly elevated B-factors, evident in both the coloration of these residues in FIG. 14A and in the plot shown in FIG. 14B; this trend was present but much more subtle in the mesotrypsin/BPTI structure, and was not recapitulated in the bovine trypsin/APPI structure. Additionally, the side chain atoms of Arg-17 showed highly elevated B-factors in the mesotrypsin/APPI structure. While it is important to keep in mind that differences in B-factors between structures reflect differences in resolution, and may also be influenced by packing arrangements in the different crystal forms, the observations may also reveal differential residual mobility at the enzyme-inhibitor interfaces that is intrinsic to the different complexes. It was hypothesized that differences in flexibility and dynamics between APPI and BPTI contribute to the observed differences in scaffold-dependent vulnerability to proteolysis by mesotrypsin.

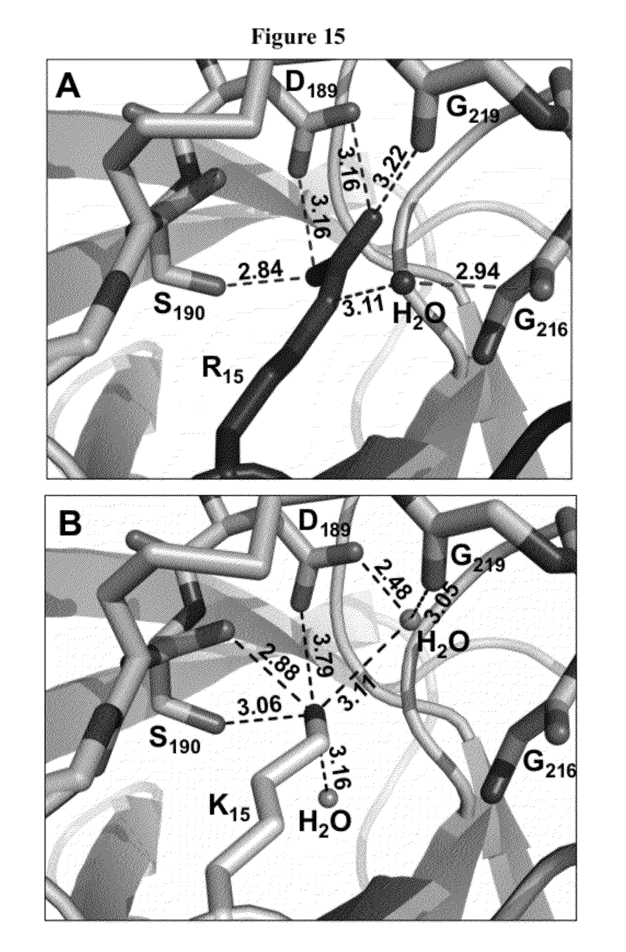

Next, mesotrypsin/APPI and mesotrypsin/APPI-R15K structures were compared, focusing on the P₁ residue that differs between these variants. The two structures showed differences in electrostatic interactions in the primary specificity pocket that may help to explain the impact of P₁ Arg vs. Lys on binding and hydrolysis (FIG. 15). The P₁ Arg residue of wt APPI appeared to form a stronger salt bridge with mesotrypsin Asp-189, with both guanidine nitrogens interacting directly with the Asp-189 carboxylate oxygens with distances of 3.16 Å (FIG. 15A). By contrast, in the mesotrypsin/APPI-R15K structure, the N_{ζ} of Lys-15 formed a much longer interaction with Asp-189 O_{δ1} (3.79 Å) and a water-bridged interaction with O_{δ2} (FIG. 15B). Thus, it was hypothesized that stronger electrostatic interactions in the S₁ site favored the binding of P₁ Arg variants.

Taken together, the binding and structural results described herein demonstrate that the affinities and proteolytic stabilities of Kunitz inhibitors towards mesotrypsin are largely orthogonal properties that can be independently manipulated. The canonical binding loop, and specifically the identity of P₁ and P₂′ residues, were found to play a dominant role in modulating binding affinity toward mesotrypsin. In addition, the identity of the scaffold beyond the canonical binding loop, due at least in part to dynamical properties, was found to be a determinant of proteolytic resistance to mesotrypsin, such that the BPTI scaffold offers a relatively stable scaffold upon which to base mesotrypsin inhibitors, whereas the APPI scaffold is comparatively vulnerable to proteolysis. In the BPTI-K15R/R17M double mutant, the higher affinity of APPI was effectively transplanted onto the BPTI scaffold through mutation of the binding loop, but nearly all of the proteolytic stability of BPTI was retained, demonstrating that one can generate stable, high affinity mesotrypsin inhibitors through engineering the canonical binding loop of a stable Kunitz scaffold to optimize affinity towards mesotrypsin.

### Example 7

### Identify Inhibitors with Enhanced Mesotrypsin Affinity and Selectivity

Site-directed mutagenesis of APPI and BPTI was performed to modulate mesotrypsin affinity. Due to the impact of mutating the P₂′ residue observed in Example 6 above, a broader range of P₂′ residues were tested in this study. The P₂′ variants span a 200-fold range of affinities (Table 7); the APPI-M17G mutant, with Kᵢ value of 4.7 nM, was the tightest-binding mesotrypsin ligand and a 30-fold improvement over wt APPI. By mutating the binding loop of BPTI to match the sequence of APPI-M17G, an inhibitor, BPTI-K15R/R17G was generated, with 1,400-fold improved mesotrypsin affinity (Ki=10 nM) relative to wt BPTI (Ki=14 μM). This inhibitor combined strong mesotrypsin affinity with proteolytic stability, and can serve as a starting point for further optimization. Preliminary studies with APPI and BPTI revealed that resistance to proteolysis and binding affinity are orthogonal properties that were independently manipulated and provided strong evidence for the feasibility of engineering a Kunitz inhibitor as a mesotrypsin-targeted therapeutic. Influence of mesotrypsin Arg-193 may shape atypical binding preferences toward additional subsites of the inhibitor's binding loop.

TABLE 7
Inhibitors.

| Inhibitor | P₁ | P₂′ | k_{cat} (s⁻¹) | Kᵢ (M) | 1/Kᵢ | k_{cat}/Kᵢ (s⁻¹M⁻¹) |
|---|---|---|---|---|---|---|
| APPI-wt | R | M | 4.22E−02 | 1.36E−07 | 7.35E+06 | 3.11E+05 |
| APPI-R15K | K | M | 3.21E−03 | 3.50E−07 | 2.86E+06 | 9.17E+03 |
| APPI-M17R | R | R | 4.57E−02 | 9.20E−07 | 1.09E+06 | 4.97E+04 |
| APPI-R15K/M17R | K | R | 3.37E−03 | 1.53E−06 | 6.54E+05 | 2.20E+03 |
| APPI-M17G | R | G | 2.36E−02 | 4.7E−09 | 2.13E+08 | 5.01E+06 |
| APPI-M17A | R | A | 2.47E−02 | 1.713E−08 | 5.8E+07 | 1.44E+06 |
| APPI-M17Y | R | Y | 1.50E−02 | 5.325E−08 | 1.88E+07 | 2.80E+05 |
| APPI-M17F | R | F | 3.42E−02 | 1.26E−07 | 7.93E+06 | 2.7E+05 |
| APPI-M17R | R | R | 4.57E−02 | 9.20E−07 | 1.087E+06 | 4.97E+04 |
| APPI-M17D | R | D | 7.11E−01 | 3.46E−07 | 2.89E+06 | 2.05E+06 |
| APPI-M17E | R | E | 6.72E−01 | 3.085E−07 | 3.24E+06 | 2.20E+06 |
| BPTI-wt | K | R | 1.87E−04 | 1.35E−05 | 7.41E+04 | 1.38E+01 |
| BPTI-K15R | R | R | 1.10E−03 | 2.40E−06 | 4.17E+05 | 4.58E+02 |
| BPTI-R17M | K | M | 1.12E−04 | 7.60E−07 | 1.32+E06 | 1.47E+02 |
| BPTI-K15R/R17G | R | G | 5.1E−05 | 1.07E−08 | 9.3E+7 | 4.76E+03 |
| BPTI-K15R/R17M | R | M | 5.14E−04 | 2.10E−07 | 4.76E+06 | 2.45E+03 |
| BPTI-K15R/R17D | R | D | 4.6E−03 | 1.5E−06 | 6.67E+05 | 3.066E+03 |

In table 7, the column marked k_{cat} reveals the rate of catalytic cleavage by mesotrypsin with smaller numbers indicating greater proteolytic stability of the variant. The column marked Kᵢ provides the inhibition constant, an indication of affinity, with smaller numbers indicating greater affinity. The BPTI-K15R/R17G was an effective inhibitor, with very low Kᵢ, and also very low k_{cat}. The APPI-M17G exhibited about 2× further improved affinity over BPTI-K15R/R17G, but exhibited a k_{cat} of 2.36E-02, indicating a much greater vulnerability to proteolysis.

Based on the large improvements in mesotrypsin binding affinity seen in BPTI variants mutated at the P₁ and P₂′ positions of the canonical binding loop, and upon the crystal structures of mesotrypsin bound to BPTI and APPI, it is anticipated that further improvements in binding affinity may be achieved through mutation of additional residues of the canonical binding loop and secondary binding loop; specifically, BPTI residues at the P₁′, P₃′, and P₄′ positions, and residue 34, which contacts both the P₂′ residue of BPTI and the enzyme. Phage display is used to comprehensively optimize the binding loop for affinity to mesotrypsin. Using BPTI-K15R/R17G as a template, a phage display library is generated in the phagemid vector pComb3H-BPTI; this construct enables monovalent display at the N-terminus of the M13 minor coat protein pIII, a strategy which allows better discrimination among tight-binding ligands as compared with polyvalent display systems (Williams et al., Transfus. Apher. Sci., 29(3):255-258 (2003); Kiczak et al., Biochim. Biophys. Acta, 1550(2):153-163 (2001); Roberts et al., Proc. Natl. Acad. Sci. USA, 89(6):2429-2433 (1992); Dennis et al., J. Biol. Chem., 270(43):25411-25417 (1995); Dennis and Lazarus, J. Biol. Chem. 269(35):22137-22144 (1994); Dennis and Lazarus, J. Biol. Chem., 269(35):22129-22136 (1994); Markland et al., Biochemistry, 35(24):8058-8067 (1996); and Markland et al., Biochemistry, 35(24):8045-8057 (1996)). The non-prime side residues of the binding loop are held invariant, while residues 16, 17, 18, and 19, representing the P₁′, P₂′, P₃′, and P₄′ positions, and residue 34, which contacts both the P₂′ residue of BPTI and the enzyme, are fully degenerated. This cloned library was constructed and contains 2.9×10⁷ independent variants, giving full coverage of all possible sequence combinations. Selection involves panning against mesotrypsin coupled to agarose beads or microtiter plates as described elsewhere (Kiczak et al., Biochim. Biophys. Acta, 1550(2):153-163 (2001); Roberts et al., Proc. Natl. Acad. Sci. USA, 89(6):2429-2433 (1992); Dennis et al., J. Biol. Chem., 270(43):25411-25417 (1995); Dennis and Lazarus, J. Biol. Chem. 269(35):22137-22144 (1994); Dennis and Lazarus, J. Biol. Chem., 269(35):22129-22136 (1994); Markland et al., Biochemistry, 35(24):8058-8067 (1996); and Markland et al., Biochemistry, 35(24):8045-8057 (1996)). After several rounds of panning and amplification, selected clones are sequenced and analyzed for preferred residues found in the degenerated positions. Once an optimized consensus sequence or several variations are identified, these sequences are grafted onto the preferred Kunitz scaffold; soluble recombinant protein is produced and purified, and Kᵢ and k_{cat} constants are determined Potential off-target effects of the engineered inhibitors on coagulation in vitro are assessed using aprotinin as a reference agent (Devy et al., Neoplasia, 9(11):927-937 (2007)).

### Example 8

### P2′ Residue is Involved in Mesotrypsin Specificity

The following is based on further analysis of data provided in the other Examples as well as additional data provided in this Example.

### Production of Recombinant Proteins

Recombinant human mesotrypsinogen, human cationic trypsinogen, and human anionic trypsinogen as well as a catalytically inactive S195A mutant of mesotrypsinogen were expressed in E. coli, isolated from inclusion bodies, refolded, purified, and activated with bovine enteropeptidase as described elsewhere (Salameh et al., J. Biol. Chem., 283:4115-4123 (2008); Sahin-Toth, J. Biol. Chem., 275:22750-22755 (2000); and Kukor et al., Eur. J. Biochem., 270:2047-2058 (2003)). Kunitz domain inhibitors were expressed in the methylotrophic yeast Pichia pastoris under control of the alcohol oxidase (AOXI) promoter using the expression vector pPICZαA (Invitrogen). Constructs containing APPI-WT, BPTI-WT, and several mutant inhibitors as well as the expression and purification of APPI-WT, BPTI-WT, and several mutant inhibitors was described elsewhere (Salameh et al., J. Biol. Chem., 285:1939-1949 (2010); Salameh et al., J. Biol. Chem., 285:36884-36896 (2010); Navaneetham et al., J. Biol. Chem., 280:36165-36175 (2005); and Navaneetham et al., J. Biochem., 148:467-479 (2010)). Additional mutations were introduced using the QuikChange kit (Stratagene), and sequence verification and expression screening were conducted as described elsewhere (Salameh et al., J. Biol. Chem., 285:36884-36896 (2010)).

### Inhibition Studies

Mesotrypsin, cationic trypsin, and anionic trypsin concentrations were quantified by active-site titration using 4-nitrophenyl 4-guanidinobenzoate (Sigma), and APPI and BPTI variant concentrations were determined by titration with bovine trypsin (Sigma) as previously elsewhere (Salameh et al., J. Biol. Chem., 283:4115-4123 (2008)). Concentrations of the chromogenic substrate benzyloxycarbonyl-Gly-Pro-Arg-p-nitroanalide (Z-GPR-pNA) (Sigma) were determined by end point assay. For determination of mesotrypsin inhibition constants, enzyme assays performed at 37° C. in the presence of varying concentrations of substrate and inhibitor were followed spectroscopically for 3-5 minutes, and initial rates were determined from the absorbance increase caused by the release of p-nitroaniline (ε₄₁₀=8480 M⁻¹ cm⁻¹) as described elsewhere (Salameh et al., J. Biol. Chem., 283:4115-4123 (2008)). Data were globally fitted by multiple regression to the classic competitive inhibition equation (equation 1), using Prism (GraphPad Software, San Diego Calif.). Reported inhibition constants are average values obtained from multiple independent experiments.

For measurement of inhibition constants with cationic and anionic trypsins, the observation of slow, tight-binding behavior required an alternative kinetic treatment, using methods described elsewhere (Salameh et al., J. Biol. Chem., 283:4115-4123 (2008)). Reactions were run at 25° C., and were followed spectroscopically for 16 hours so that reliable steady-state rates could be obtained. Inhibition constants were calculated using equation 2 as described elsewhere (Salameh et al., J. Biol. Chem., 283:4115-4123 (2008)), where vᵢ and v₀ are the steady-state rates in the presence and absence of inhibitor, K_{M} is the Michaelis constant for substrate cleavage, and [S₀] and [I₀] are the initial concentrations of substrate and inhibitor. Calculations were performed using K_{M} values of 36.5 μM for cationic trypsin and 22.6 μM for anionic trypsin, determined from Michaelis-Menten kinetic studies.

(v₀−vᵢ)/vᵢ=[I₀]/Kᵢ(1+[S₀]/Kₘ) (2)

### Impact of P₂′ Residues on Free Energies of Association, Catalysis, and Transition State Stabilization

Using the absence of a P₂′ side chain in APPI-M17G as the baseline for comparison of all other variants, and using 1/Kᵢ as an approximation of Kₐ, the equilibrium association constant, the change in free energy of association ΔΔGₐ° (Gly17X) was calculated from equation 3. Changes in the free energy of catalysis ΔΔG_{cat}(Gly17X) and in the transition-state stabilization energy ΔΔG_{T}^{‡}(Gly17X) were similarly calculated from equations 4 and 5, respectively.

$$\begin{matrix}ΔΔ {G}_{a}^{°} = - RT ln \frac{{( {K}_{a} )}_{APPI \text{-} X 17}}{{( {K}_{a} )}_{APPI \text{-} Gly 17}} & ( 3 ) \\ ΔΔ {G}_{cat} = - RT ln \frac{{( {k}_{cat} )}_{APPI \text{-} X 17}}{{( {k}_{cat} )}_{APPI \text{-} Gly 17}} & ( 4 ) \\ ΔΔ {G}_{T}^{‡} = - RT ln \frac{{( {k}_{cat} / {K}_{m} )}_{APPI \text{-} X 17}}{{( {k}_{cat} / {K}_{m} )}_{APPI \text{-} Gly 17}} & ( 5 )\end{matrix}$$

### Inhibitor Hydrolysis Studies

The depletion of intact APPI and BPTI variants in time course incubations with active mesotrypsin was monitored by SDS-PAGE and HPLC as described elsewhere (Salameh et al., J. Biol. Chem., 283:4115-4123 (2008); Salameh et al., J. Biol. Chem., 285:1939-1949 (2010); and Salameh et al., J. Biol. Chem., 285:36884-36896 (2010)). SDS-PAGE was used to obtain initial qualitative estimates reaction rates, while HPLC was used to quantitatively determine catalysis rates (k_{cat}). Incubations of mesotrypsin with BPTI mutants were carried out in 0.1 M Tris-HCl pH 8.0 and 1 mM CaCl₂ at 37° C. BPTI variant concentration was 50 μM, and mesotrypsin concentration was 1-5 μM. Aliquots for HPLC analysis were withdrawn from BPTI hydrolysis reactions at periodic intervals, adjusted to 6 M urea and 2 mM DTT, incubated for 10 minutes at 37° C., quenched by acidification to pH 1, and then frozen at −20° C. until as described elsewhere (Salameh et al., J. Biol. Chem., 283:4115-4123 (2008)). Incubations of mesotrypsin with APPI mutants were carried out similarly, except that APPI concentration was 50 μM and mesotrypsin concentration was in the range of 10-500 nM. APPI hydrolysis time point samples were not denatured or reduced. Instead, samples were quenched immediately by acidification to pH≦1 and then frozen at −20° C. until analyzed as described elsewhere (Salameh et al., J. Biol. Chem., 285:1939-1949 (2010); and Salameh et al., J. Biol. Chem., 285:36884-36896 (2010)). Enzyme, Kunitz inhibitors, and hydrolysis products were resolved by HPLC, and the disappearance of intact Kunitz inhibitors over time was quantified by peak integration as described elsewhere (Salameh et al., J. Biol. Chem., 283:4115-4123 (2008); Salameh et al., J. Biol. Chem., 285:1939-1949 (2010); and Salameh et al., J. Biol. Chem., 285:36884-36896 (2010)). Initial rates were obtained by linear regression using a minimum of 5 data points within the initial linear phase of the reaction, and not exceeding 50% conversion of intact inhibitor to hydrolysis products. Hydrolysis rates reported for each inhibitor represent the average of 2-3 independent experiments.

### Crystalization of a Mesotrypsin•BPTI-K15R/R17D Variant Complex

The catalytically inactive mesotrypsin-S195A mutant was mixed with BPTI-K15R/R17D in a 1:1 stoichiometric molar ratio. The heterodimeric complexes were further purified by HiLoad Superdex 75 gel filtration chromatography (GE Healthcare), exchanged into 10 mM Na acetate, pH 6.5, and concentrated to ˜3 mg/mL. Complexes were crystallized at 22° C. in hanging drops, over a reservoir of 25% PEG4000, 0.2 M Na acetate and 100 mM Tris, pH 8.0. Drops (4 μL) were prepared by mixing equal volumes of protein and reservoir solutions. Crystals (0.2×0.4×0.1 mm) appeared within 2 days and grew over the course of 6 days. Crystals were harvested, soaked in a cryoprotectant solution (30% PEG4000, 0.2 M Na acetate, 100 mM Tris pH 8.0 and 15% glycerol) and flash-frozen in liquid N₂.

### X-Ray Data Collection, Structure Solution, and Model Refinement

Synchrotron X-ray data were collected from crystals at 100 K using ADSC CCD detectors at beam lines X12-B, X12-C, and X25 at the National Synchrotron Light Source, Brookhaven National Laboratory. The software package HKL2000 (Otwinowski, Z. and Minor, W. (1997) Processing of X-ray Diffraction Data Collected in Oscillation Mode. In Macromolecular Crystallography, part A (Carter, C. W., Jr. and Sweet, R. M., eds.). pp. 307-326, Academic Press, New York) was used for integrating, scaling and merging the reflection data. The structures were solved by molecular replacement using the program Phaser (McCoy et al., Acta Crystallogr. D Biol. Crystallogr., 61:458-464 (2005)) operated by PHENIX (Adams et al., Acta Crystallogr. D Biol. Crystallogr., 58: 1948-1954 (2002)), using the mesotrypsin•BPTI-WT complex structure (PDB ID 2R9P) (Salameh et al., J. Biol. Chem., 283:4115-4123 (2008)) as the search model. The successful solution contained one copy of the heterodimeric complex in the asymmetric unit. Cycles of manual rebuilding in COOT (Emsley and Cowtan, Acta Crystallogr. D Biol. Crystallogr., 60:2126-2132 (2004)) were alternated with automated refinement using the refinement module of the PHENIX software suite (Afonine et al., The Phenix refinement framework. CCP4 Newsletter. 42 (2005)). A test set comprised of 10% of the total reflections was excluded from refinement to allow calculation of the free R factor. Waters, ions, and alternative conformations of protein residues were added using COOT (Emsley and Cowtan, Acta Crystallogr. D Biol. Crystallogr., 60:2126-2132 (2004)). At early stages, TLS refinement was employed with each protein chain assigned to a separate TLS group. At later stages of refinement, full anisotropic treatment of atomic displacement parameters was employed, and hydrogen atoms were added in the riding positions. All superpositions and structure figures were created using the graphics software Pymol (http://pymol.sourceforge.net).

### Results

### The P₂′ Position Plays a Role in Mesotrypsin Substrate Binding Affinity

Replacement of the bulky hydrophobic Met, which occupies the P₂′ position of APPI-WT, with small (Gly, Ala), aromatic (Phe, Tyr), basic (Arg), or acidic (Asp, Glu) amino acids produced a spectrum of Kᵢ values spanning over two orders of magnitude, illustrating the importance of this position in mesotrypsin binding discrimination. The tightest binding of the APPI variants tested was APPI-M17G, which completely lacks a side chain at the P₂′ position and possesses a Kᵢ value toward mesotrypsin of 4.7 nM. APPI-M17G was considered as a baseline for assessing the impact of the various other P₂′ side chains on mesotrypsin association. Using 1/Kᵢ as an approximation of Kₐ, the equilibrium association constant, the change in free energy of association ΔΔGₐ° (Gly17X) was calculated. These ΔΔGₐ° values are listed in Table 8.

TABLE 8
Strength of association of mesotrypsin with APPI variants.

| | | | | ΔΔGₐ° |
|---|---|---|---|---|
| | | Kᵢ ± SD | Relative Kᵢ | (Gly17X) |
| Inhibitor | P₂′ | (M) | (fold diff) | (kcal/mol) |
| APPI-M17G | G | 4.71 ± 0.13 × 10⁻⁹ | 1.0 | 0.00 |
| APPI-M17A | A | 1.71 ± 0.27 × 10⁻⁸ | 3.6 | 0.80 |
| APPI-WT* | M | 1.36 ± 0.19 × 10⁻⁷ | 28.9 | 2.07 |
| APPI-M17F | F | 1.26 ± 0.11 × 10⁻⁷ | 26.8 | 2.02 |
| APPI-M17Y | Y | 5.33 ± 0.32 × 10⁻⁸ | 11.3 | 1.49 |
| APPI-M17R* | R | 9.20 ± 0.99 × 10⁻⁷ | 195.3 | 3.25 |
| APPI-M17D | D | 3.46 ± 0.18 × 10⁻⁷ | 73.5 | 2.65 |
| APPI-M17E | E | 3.09 ± 0.18 × 10⁻⁷ | 65.5 | 2.58 |

*Kinetic constants reported elsewhere (Salameh et al., J. Biol. Chem., 285: 1939-1949 (2010); and Salameh et al., J. Biol. Chem., 285: 36884-36896 (2010)).

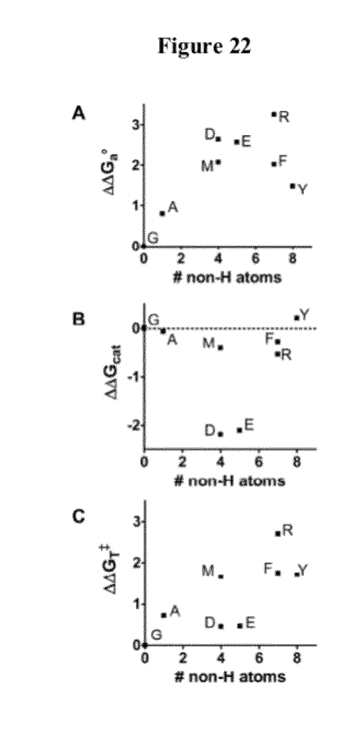

It appears that P₂′ side chains are deleterious for binding by comparison with Gly. When ΔΔGₐ° (Gly17X) values were plotted vs. the number of non-hydrogen atoms in each side chain (Bigler et al., Protein Sci., 2:786-799 (1993) and Lu et al., J. Mol. Biol., 266:441-461 (1997)), there is a clear trend toward weaker binding (larger ΔΔGₐ°) with increasing steric bulk of the P₂′ residue (FIG. 22A). Side chain charge appears to contribute to a deleterious effect on binding as well, since variants possessing acidic (Asp, Glu) or basic (Arg) side chains each display higher ΔΔGₐ° than those featuring uncharged residues of similar size.

### Acidic Residues at the P₂′ Position Accelerate Mesotrypsin-Catalyzed Hydrolysis

HPLC assays were used to directly quantify cleavage of APPI variants by mesotrypsin in time course incubations (Salameh et al., J. Biol. Chem., 285:1939-1949 (2010); and Salameh et al., J. Biol. Chem. 285, 36884-36896 (2010)). Because hydrolysis studies used APPI concentrations >50-fold higher than Kₘ values, mesotrypsin binding capacity was saturated, and observed rates of hydrolysis approximated true catalytic rate constants (k_{cat}). Values of k_{cat} and specificity constant k_{cat}/Kₘ for each APPI variant considered as a substrate for mesotrypsin are summarized in Table 9. APPI-M17G was used as a baseline for comparison, and changes in the free energy of catalysis ΔΔG_{cat}(Gly17X) and in transition-state stabilization energy ΔΔG_{T}^{‡}(Gly17X) reflecting contributions to catalysis of each non-Gly P₂′ residue tested were calculated.

TABLE 9
Catalytic cleavage of APPI variants by mesotrypsin.

| | | k_{cat} ± SD | Rel k_{cat} | ΔΔG_{cat} | k_{cat}/Kₘ ± SD | k_{cat}/Kₘ | ΔΔG_{T}^{‡} |
|---|---|---|---|---|---|---|---|
| Inhibitor | P₂′ | (s⁻¹) | (fold diff) | (kcal/mol) | (s⁻¹M⁻¹) | (fold diff) | (kcal/mol) |
| APPI-M17G | G | 2.22 ± 0.13 × 10⁻² | 1.0 | 0.00 | 4.71 ± 0.31 × 10⁶ | 1.00 | 0.00 |
| APPI-M17A | A | 2.45 ± 0.14 × 10⁻² | 1.1 | −0.06 | 1.43 ± 0.24 × 10⁶ | 0.30 | 0.73 |
| APPI-WT* | M | 4.32 ± 0.40 × 10⁻² | 1.9 | −0.41 | 3.18 ± 0.53 × 10⁵ | 0.067 | 1.66 |
| APPI-M17F | F | 3.44 ± 0.25 × 10⁻² | 1.6 | −0.27 | 2.73 ± 0.31 × 10⁵ | 0.058 | 1.75 |
| APPI-M17Y | Y | 1.57 ± 0.07 × 10⁻² | 0.71 | 0.21 | 2.95 ± 0.22 × 10⁵ | 0.063 | 1.71 |
| APPI-M17R* | R | 5.36 ± 0.16 × 10⁻² | 2.4 | −0.54 | 5.83 ± 0.65 × 10⁴ | 0.012 | 2.71 |
| APPI-M17D | D | 7.64 ± 0.76 × 10⁻¹ | 34.4 | −2.18 | 2.21 ± 0.25 × 10⁶ | 0.47 | 0.47 |
| APPI-M17E | E | 6.72 ± 0.70 × 10⁻¹ | 30.3 | −2.10 | 2.18 ± 0.26 × 10⁶ | 0.46 | 0.48 |

*Salameh et al., J. Biol Chem., 285: 1939-1949 (2010); and Salameh et al., J. Biol Chem., 285: 36884-36896 (2010).

Mutations at the P₂′ position generally appeared to have very little impact on mesotrypsin catalytic rate constants, irrespective of residue size (FIG. 22B). The only exceptions noted were for replacement of the P₂′ amino acid with acidic residues Asp or Glu, which both accelerated k_{cat} by ˜30-fold, contributing ˜−2 kcal/mol to the free energy of catalysis. Since most P₂′ residues had substantial impact on Kₘ but negligible effect on k_{cat}, the impact on specificity constant k_{cat}/Kₘ was dominated by Kₘ effects, and a plot of ΔΔG_{T}^{‡}(Gly17X) showed positive (deleterious) changes in transition-state stabilization energy trending upward with residue size (FIG. 22C). Asp and Glu diverged from this pattern. For these residues, the deleterious effect of the acidic side chain on binding was substantially offset by the favorable impact on catalytic rate.

Optimization of the P₂′ Residue Converts BPTI into a Potent Mesotrypsin Inhibitor

It was hypothesized that P₂′ mutations characterized in the context of APPI would have parallel effects on the binding affinity and vulnerability to mesotrypsin hydrolysis of BPTI, and that this information might be used to engineer BPTI as a more potent inhibitor of mesotrypsin. Starting with the BPTI-K15R variant, which possesses a P₁ Arg residue and binds 6-fold more tightly to mesotrypsin than BPTI-WT, either Gly or Asp was introduced at the P₂′ position. These new mutants, along with BPTI-K15R and BPTI-K15R/R17M variants, allowed for a comparison of the impact of an absent (Gly), hydrophobic (Met), basic (Arg), or acidic (Asp) side chain at the BPTI P₂′ position. For each variant, Kᵢ was obtained from competitive inhibition experiments and k_{cat} derived from HPLC-based hydrolysis studies as described elsewhere (Salameh et al., J. Biol. Chem., 283:4115-4123 (2008) and Salameh et al., J. Biol. Chem., 285:36884-36896 (2010)). The BPTI variant with P₂′ Gly was used as a baseline for comparison, allowing evaluation of the energetic contributions of Met, Arg, or Asp side chains to mesotrypsin binding and catalysis.

Comparing the Kᵢ values for mesotrypsin obtained with the BPTI variants (Table 10), it was apparent that as in the case of the substrate APPI, all tested non-Gly residues at the P₂′ position proved deleterious for binding. The rank order of BPTI variants ranked according to Kᵢ matched that observed for the APPI variants: Gly exhibited the tightest association, followed by Met, Asp, and then Arg, suggesting that for BPTI as for APPI, the bulk and charge of the P₂′ residue were important determinants of binding specificity. Mutation of the P₂′ residue to Gly in BPTI-K15R/R17G produced the tightest-binding inhibitor of mesotrypsin reported to date, with a Kᵢ value of 5.9 nM, over 2000-fold lower than that of BPTI-WT, and over 400-fold lower than that of BPTI-K15R mutated only at the P₁ position, another strong determinant of mesotrypsin binding specificity.

TABLE 10
Strength of association of mesotrypsin with BPTI variants.

| | | | | ΔΔGₐ° |
|---|---|---|---|---|
| | | Kᵢ ± SD | Relative Kᵢ | (Gly17X) |
| Inhibitor | P₂′ | (M) | (fold diff) | (kcal/mol) |
| BPTI-K15R/R17G | G | 5.9 ± 1.7 × 10⁻⁹ | 1.0 | 0.00 |
| BPTI-K15R/R17M* | M | 2.18 ± 0.01 × 10⁻⁷ | 36.7 | 2.22 |
| BPTI-K15R* | R | 2.38 ± 0.20 × 10⁻⁶ | 401.6 | 3.69 |
| BPTI-K15R/R17D | D | 1.50 ± 0.09 × 10⁻⁶ | 252.4 | 3.41 |

*Salameh et al., J. Biol Chem., 285: 36884-36896 (2010).

An effective polypeptide inhibitor should resist inactivation and degradation through proteolytic cleavage as well as compete effectively with substrates for binding to the active site of the protease. The k_{cat} values for mesotrypsin cleavage of BPTI variants (Table 11) revealed that the identity of the P₂′ residue has an impact on the proteolytic stability vs. vulnerability to mesotrypsin of BPTI. As in the Kᵢ comparisons, the rank orders of corresponding BPTI and APPI variants ranked according to k_{cat} remained the same, with P₂′ Gly the most resistant to proteolysis followed by Met, Arg, and then Asp, but for the BPTI variants the impact of P₂′ substitutions on k_{cat} was magnified. In particular, the P₂′ Asp variant BPTI-K15R/R17D displayed a 90-fold enhanced catalytic rate relative to the P₂′ Gly variant. Notably, the P₂′ Gly variant was an effective inhibitor both from the viewpoint of binding affinity and from the viewpoint of proteolytic stability to mesotrypsin. Table 11 also provides k_{cat}/Kₘ substrate specificity constants for each BPTI variant considered as a substrate rather than an inhibitor of mesotrypsin. All of the BPTI variants featured specificity constants 2-3 orders of magnitude lower than those seen for the corresponding APPI variants in Table 9 as a consequence of their much slower rates of cleavage by mesotrypsin. This resistance to proteolysis was an attribute conferred by the BPTI scaffold.

TABLE 4
Catalytic cleavage of BPTI variants by mesotrypsin

| | | k_{cat} ± SD | Rel k_{cat} | ΔΔG_{cat} | k_{cat}/Kₘ ± SD | k_{cat}/Kₘ | ΔΔG_{T}^{‡} |
|---|---|---|---|---|---|---|---|
| Inhibitor | P₂′ | (s⁻¹) | (fold diff) | (kcal/mol) | (s⁻¹M⁻¹) | (fold diff) | (kcal/mol) |
| BPTI-K15R/R17G | G | 5.09 ± 0.88 × 10⁻⁵ | 1.0 | 0.00 | 8.6 ± 2.9 × 10³ | 1.00 | 0.00 |
| BPTI-K15R/R17M* | M | 5.14 ± 0.23 × 10⁻⁴ | 10.1 | −1.42 | 2.36 ± 0.11 × 10³ | 0.28 | 0.79 |
| BPTI-K15R* | R | 8.7 ± 2.7 × 10⁻⁴ | 17.0 | −1.75 | 3.6 ± 1.2 × 10² | 0.042 | 1.95 |
| BPTI-K15R/R17D | D | 4.62 ± 0.17 × 10⁻³ | 90.8 | −2.78 | 3.09 ± 0.21 × 10³ | 0.36 | 0.63 |

*Salameh et al., J. Biol Chem., 285: 36884-36896 (2010).

### Optimized BPTI Variant Retains Strong Affinity Toward Other Trypsins

To assess the potential binding selectivity of BPTI-K15R/R17G toward mesotrypsin relative to other trypsins, inhibition constants Kᵢ were measured for BPTI-WT and BPTI-K15R/R17G against recombinant human cationic trypsin and human anionic trypsin. Because the association of BPTI with these trypsins follows a slow, tight-binding model, it was necessary to use an alternative approach to the measurement of Kᵢ values as described herein. As shown in Table 12, both inhibitors bind substantially more tightly to cationic and anionic trypsin than to mesotrypsin. However, while the dual mutations introduced in BPTI-K15R/R17G exhibited an improved Kᵢ toward mesotrypsin by a factor of 2277, they exhibited a weakened binding toward cationic and anionic trypsin by a factor of 1.6-1.8. Thus, while BPTI-WT exhibited a strong selectivity toward both cationic and anionic trypsins in preference to mesotrypsin by a gap of nearly six orders of magnitude, for BPTI-K15R/R17G, this gap was narrowed to a factor of 180-200. BPTI-K15R/R17G appeared to be a relatively nonselective inhibitor with an unusual capacity to potently inhibit mesotrypsin in addition to other trypsins.

TABLE 12
Inhibition of human trypsin isoforms
by BPTI and BPTI-K15R/R17G.

| | BPTI-WT | BPTI-K15R/R17G | |
|---|---|---|---|
| Trypsin | Kᵢ ± SD (M) | Kᵢ ± SD (M) | fold |
| isoform | (SI)* | (SI)* | change |
| Mesotrypsin | 1.4 ± 0.2 × 10⁻⁵ | 5.9 ± 1.7 × 10⁻⁹ | 2277 |
| | (1) | (1) | |
| Cationic | 2.0 ± 0.1 × 10⁻¹¹** | 3.3 ± 0.1 × 10⁻¹¹ | 0.61 |
| trypsin | (1.5 × 10⁻⁶) | (5.6 × 10⁻³) | |
| Anionic | 1.7 ± 0.2 × 10⁻¹¹ | 3.0 ± 0.3 × 10⁻¹¹ | 0.55 |
| trypsin | (1.2 × 10⁻⁶) | (5.1 × 10⁻³) | |

*Selectivity Index (SI) = Kᵢ/K_{i (mesotrypsin)}
**Kᵢ Salameh et al., J. Biol. Chem. 283, 4115-4123 (2008)

Structures of Mesotrypsin in Complex with BPTI P₂′ Variants Reveal Multiple Conformations of Arg-193

To determine why the P₂′ residue played such a role in inhibitor binding and hydrolysis by mesotrypsin, the mesotrypsin•BPTI-K15R/R17G and mesotrypsin•BPTI-K15R/R17D complexes were crystallized, and their X-ray structures were solved and compared to the crystal structure of mesotrypsin bound to BPTI-WT (PDB ID 2R9P). To avoid heterogeneity associated with BPTI proteolysis, an inactive mesotrypsin-S195A mutant was used. Diffraction data were measured to 1.6 and 1.3 Å resolution for mesotrypsin•BPTI-K15R/R17G and mesotrypsin•BPTI-K15R/R17D complexes, respectively. Both crystals belonged to the space group P2₁ and exhibited very similar unit cell parameters, with a single heterodimer in the asymmetric unit. Both structures were solved by molecular replacement. Table 13 summarizes the crystal, data collection, and refinement statistics.

TABLE 6
Data collection and refinement statistics
for mesotrypsin/BPTI complexes.

| | Mesotrypsin-BPTI- | Mesotrypsin-BPTI- |
|---|---|---|
| | K15R/R17G | K15R/R17D |
| PDB ID | 3P92 | 3P95 |
| Complexes per | 1 | 1 |
| ASU | | |
| Space group | P2₁ | P2₁ |
| Unit cell, Å | 44.3, 39.2, 68.8, | 43.9, 39.1, 68.5, |
| | 90°, 100.3°, 90° | 90°, 100.1°, 90° |
| Resolution range, Å | 33.85-1.60 | 33.82-1.30 |
| Unique reflections | 27,579 | 53,441 |
| Completeness, % | 88.9 (46.7*) | 94.4 (65.0**) |
| Multiplicity | 6.5 (3.8) | 6.3 (3.8) |
| I/S.D. | 39.8 (18.4) | 17.1 (6.5) |
| R_{sym} | 0.032 (0.081) | 0.068 (0.227) |
| R_{cryst}/R_{free} | 11.13/15.86 | 11.05/13.19 |
| R.m.s.d. bonds, Å | 0.007 | 0.016 |
| R.m.s.d. angles, ° | 0.907 | 1.335 |

*Completeness at 1.85 Å is 95.1%
**Completeness at 1.43 Å is 97.1%

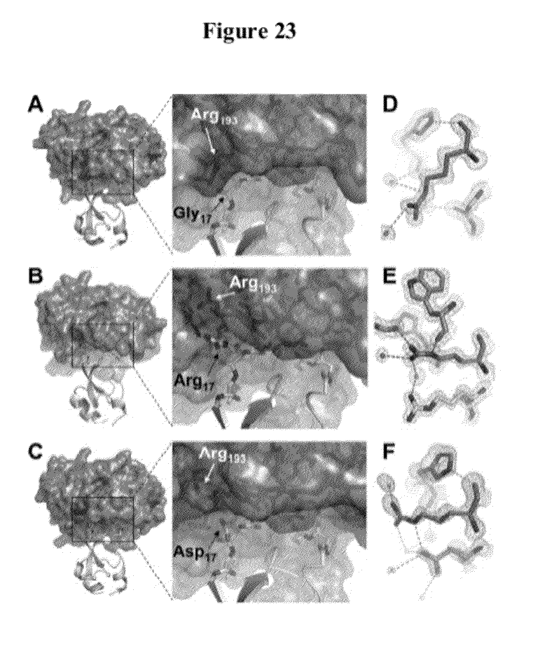

In comparing the structures of the BPTI-K15R/R17G, BPTI-K15R/R17D, and BPTI-WT complexes, it was found that while each model featured BPTI similarly bound in the canonical fashion at the mesotrypsin active site, the different P₂′ residues exhibited significant differences in interface topology (FIGS. 23A, 23B, and 23C). In the mesotrypsin•BPTI-K15R/R17G structure, Arg-193 protruded downward, enveloping the BPTI Gly-17 backbone (FIG. 23A). In this conformation, Arg-193 had 96.4 Å² of accessible surface area (ASA), 65.5 Å² (68%) of which became buried by contact with BPTI-K15R/R17G (calculations from the PDBePISA server; Krissinel and Henrick, J. Mol. Biol., 372:774-797 (2007)). By contrast, in the mesotrypsin•BPTI-WT complex, the presence of the BPTI P₂′ Arg residue displaced Arg-193 to a position buried between the two β-barrel domains of the enzyme. In this conformation, Arg-193 had only 48.9 Å² ASA, 30.2 Å² (62%) of which became buried by contact with BPTI (FIG. 23B). The mesotrypsin•BPTI-K15R/R17D complex was intermediate between these two extremes. Arg-193 adopted yet a third distinct conformation, with 62.3 Å² ASA, 30.8 Å² (49%) of which became buried by contact with BPTI (FIG. 23C).

The discrete conformations of Arg-193 found in the mesotrypsin•BPTI-K15R/R17G structure and in the mesotrypsin•BPTI-WT structure were well-ordered, as evidenced by strong electron density for the entire side chain (FIGS. 23D and 23E). The P₂′ Arg residue of BPTI-WT was well ordered as well (FIG. 23E). By contrast, the intermediate position adopted by Arg-193 in the mesotrypsin•BPTI-K15R/R17D structure, as well as the position of the P₂′ Asp-17 residue of BPTI, appeared to be somewhat less stable and less well ordered, as both Arg-193 and Asp-17 exhibited weaker electron density despite the higher resolution of this 1.3 Å structure (FIG. 23F). While N_{ε} of Arg-193 and O_{δ2} of Asp-17 were separated by only 2.75 Å in the model, the weak electron density of these side chains, and the presence of multiple weak water peaks as alternative H-bonding partners to Asp-17 O_{δ2} (FIG. 23F) suggested that the interaction between these residues may be weak and transient in nature. This structural interpretation was consistent with the biochemical data as it was found that BPTI-K15R/R17D exhibited only marginally better mesotrypsin affinity than BPTI-K15R, which features positively charged Arg-17 at the P₂′ position (Table 10). Thus, the Arg-193-Asp-17 electrostatic interaction appeared to contribute minimally to binding affinity.

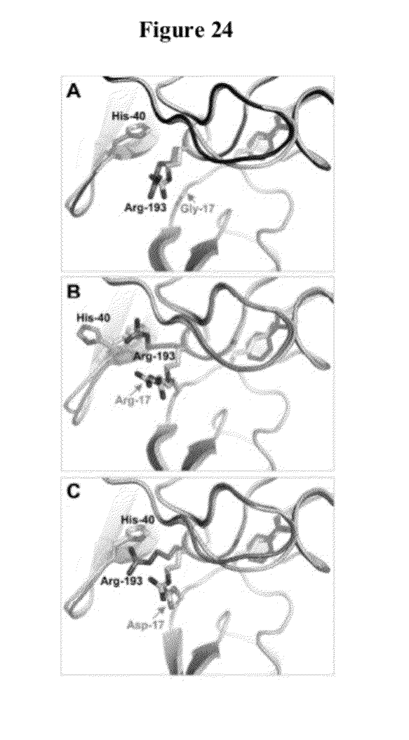

Further insights into the differential mesotrypsin affinities of BPTI P₂′ variants came from superposing the structures onto the structure of mesotrypsin bound to benzamidine (1H4W; Katona et al., J. Mol. Biol., 315:1209-1218 (2002)). Benzamidine was a small molecule that filled only the trypsin specificity pocket occupied by the P₁ Arg or Lys side chain of a substrate or polypeptide inhibitor. The mesotrypsin-benzamidine complex was expected to closely approximate the free enzyme. The superpositions revealed that very little adjustment was required of the mesotrypsin active site in order to accommodate BPTI-K15R/R17G binding. Thus, it appeared that enzyme and inhibitor are preconfigured for optimal complementarity, and that their interaction resembles lock- and-key type molecular recognition (FIG. 24A). By contrast, conformational changes involving displacement of Arg-193 in order to avoid steric clash with P₂′ Arg or Asp residues were required for binding to BPTI-WT (FIG. 14B) or BPTI-K15R/R17D (FIG. 24C). The binding data, which revealed 250-400-fold weaker binding for BPTI variants substituting P₂′ Asp or Arg for Gly (Table 3), suggested that the reorganization of the enzyme active site required to accommodate the conformational shift in Arg-193 confers an energetic penalty.

### Example 9

### Inhibiting Serine Protease-Induced Prostate Cancer Progression

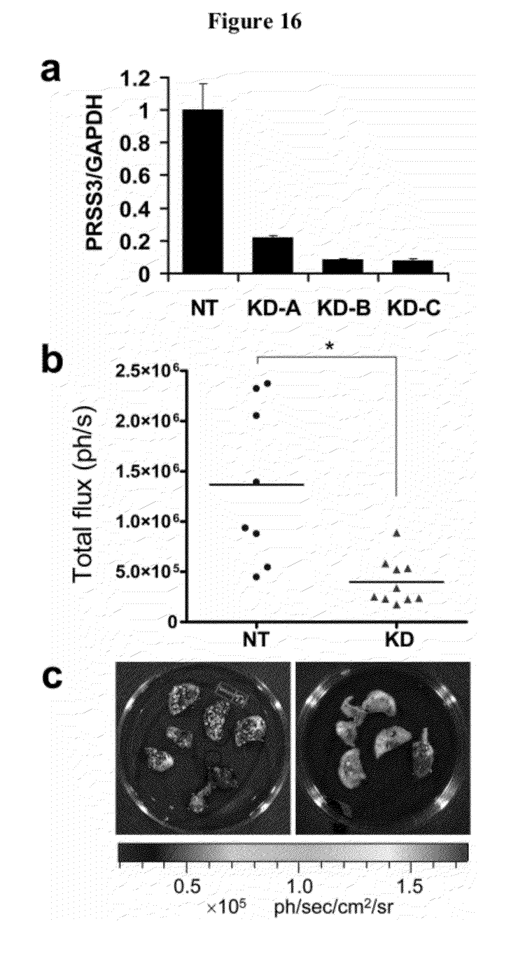

Suppression of mesotrypsin (PRSS3) expression in PC3-M cells resulted in a highly significant reduction in spontaneous metastasis to the lungs (FIG. 16). PC3-M cells were transduced with a lentiviral shRNA construct targeting PRSS3 or with a non-target control virus, and then were superinfected with a lentivirus conferring expression of firefly luciferase. Cells (1×10⁵) were implanted into the dorsolateral prostate lobe of each of 19 NOD/SCID mice (9 mice in the control group and 10 mice in the PRSS3 knockdown group). At 2 weeks post-implantation, mice were sacrificed, and metastasis was evaluated and quantified in lungs and other organs by ex vivo bioluminescent imaging. Measurement of transcript levels by qRT/PCR in several of the primary tumors from the test group confirmed that PRSS3 expression remained stably suppressed (FIG. 16A). One mouse in the control group did not have any sign of a tumor in the prostate, and was excluded from further analysis. Of the remaining eight control mice, all showed evidence of extensive metastasis to the lungs, and additionally, two had metastasis to liver and two had metastasis to spleen. Of the ten mice bearing tumors in which PRSS3 expression had been suppressed, only four showed visible evidence of lung metastasis by bioluminescent imaging, and none showed evidence of metastasis to liver or spleen. When tumor burden was assessed in the lungs as total flux of the lung lobes and the two groups were compared with a Mann Whitney analysis, it was found that the difference in mean tumor burden between the groups was highly significant (P=0.0021; FIG. 16B).

A prototype mesotrypsin inhibitor, BPTI-K15R/R17G, was generated as described herein. BPTI-K15R/R17G exhibited a 1,400-fold enhanced affinity toward mesotrypsin and possessed an inhibition constant K, of 10 nM. The proteolytic stability of BPTI-K15R/R17G to mesotrypsin digestion was such that BPTI-K15R/R17G exhibited 4-fold improved stability relative to wt BPTI, with a k_{cat} of 5.1×10⁻⁵ s⁻¹. The crystal structure of BPTI-K15R/R17G in complex with mesotrypsin revealed that mutation of the P₁ inhibitor residue to Arg resulted in a stronger stabilizing network of hydrogen bonds in the primary specificity pocket of the enzyme, while mutation of the P₂′ residue to Gly rendered the primed side of the inhibitor binding loop much more sterically complementary to the unbound state of the enzyme.

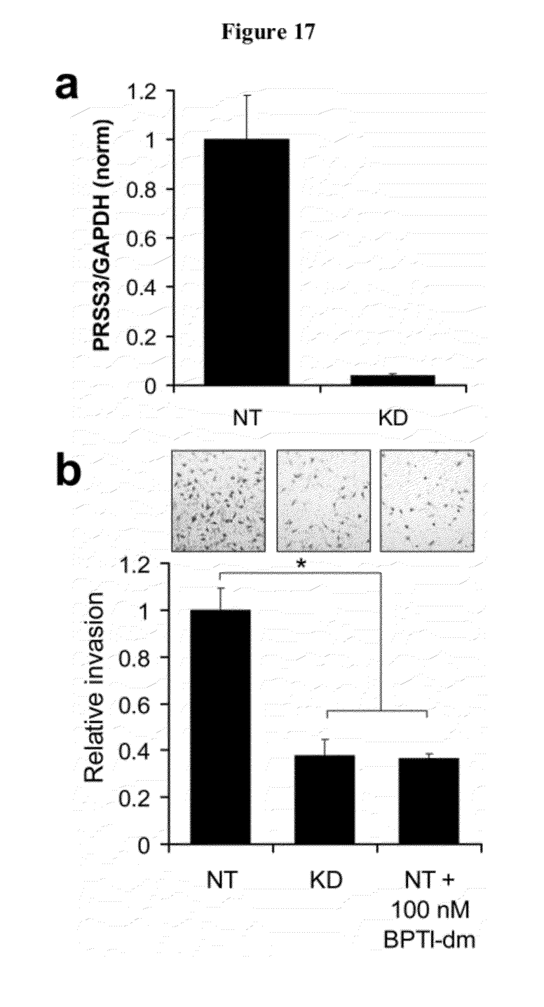

Pharmacologic inhibition of mesotrypsin inhibited PC3-M prostate cancer cell invasion (FIG. 17). Cells (2×10⁴) were plated in triplicate in BD Biocoat Matrigel transwell invasion chambers in serum-free RPMI medium containing 0.1% BSA, and allowed to invade for 18 hours toward NIH/3T3 cell conditioned medium in the lower chamber. In some wells, cells were treated with 100 nM BPTI-K15R/R17G during the course of the invasion assay; this inhibitor concentration, 10-fold higher than the inhibition constant (Kᵢ) toward mesotrypsin, was chosen to ensure effective quenching of mesotrypsin activity under the assay conditions. Filters were fixed with methanol, stained with crystal violet, photographed, and then invasive cells were counted using automated ImagePro software. In this study, efficient suppression of mesotrypsin expression by shRNA knockdown (FIG. 17A) resulted in a 63% reduction in the number of invading cells. Pharmacologic inhibition of mesotrypsin by BPTI-K15R/R17G resulted in a nearly identical reduction in invasion (FIG. 17B). These results demonstrate that improved mesotrypsin inhibitors can be obtained through bioengineering of the Kunitz scaffold and that such inhibitors can be used to inhibit mesotrypsin in vivo and treat conditions such as cancer.

### Example 10

### Inhibiting Breast Cancer Cell Malignant Growth

Whereas HMT3522 T4-2 malignant human breast cells proliferate into tumor-like masses when grown in a 3D laminin-rich reconstituted basement membrane gel, suppression of mesotrypsin (PRSS3) expression in these cells by transduction with an shRNA construct targeting PRSS3 resulted in a phenotypic reversion to a less malignant phenotype. T4-2 cells with inhibition of PRSS3 expression showed suppression of disorganized malignant growth and enhanced formation of acini with basal polarity (Hockla et al., Breast Cancer Res. Treat., 124(1):27-38 (2010)).

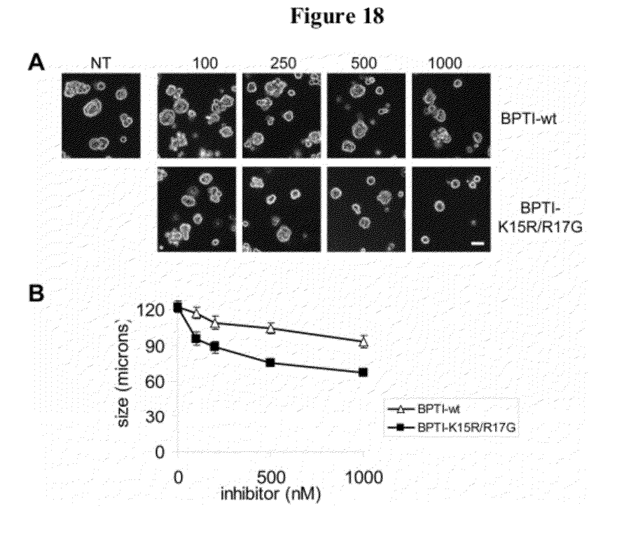

The prototype mesotrypsin inhibitor described herein, BPTI-K15R/R17G, was used to test the effect of pharmacologic inhibition of mesotrypsin in T4-2 malignant breast cells. T4-2 cells were grown in Matrigel for 7 days as described elsewhere (Hockla et al., Breast Cancer Res. Treat., 124(1):27-38 (2010)), in the presence of 100 nM, 200 nM, 500 nM, or 1000 nM BPTI-wt or BPTI-K15R/R17G inhibitors, and then photographed and assessed for colony size. Photographs of representative fields revealed an inhibitor concentration-dependent reduction of colony size compared to control cultures (FIG. 18A). Colony size (obtained from morphometric measurement of 30 colonies per condition) was significantly reduced by both inhibitors; however, the BPTI-K15R/R17G mutant was more than 10-fold more potent, as colony growth with 100 nM BPTI-K15R/R17G was further reduced compared with cultures treated with 1000 nM BPTI-wt.

### Example 11

### BPTI-K15R/R17G Inhibits Pancreatic Cancer Cell Malignant Growth and Invasion

Capan-1 human pancreatic cancer cells (obtained from Dr. P. Storz, Mayo Clinic) were cultured in DMEM supplemented with 10% fetal bovine serum. Cells were transduced with lentiviral short hairpin RNA construct NM_{—}002771.2-454s1c1 targeting human PRSS3, from the MISSION TRC-Hs1.0 library (Sigma), or with a negative control vector containing a short hairpin that does not recognize any human genes as described elsewhere (Hockla et al., Breast Cancer Res. Treat., 124:27-38 (2010)). After selection with 1 μg/mL puromycin, pooled transductants were split into three parallel cultures for (a) analysis of PRSS3 transcript levels by qRT/PCR as described elsewhere (Hockla et al., Breast Cancer Res. Treat., 124:27-38 (2010)), (b) analysis of mesotrypsin protein levels, and (c) assessment of cellular invasiveness. Knockdown was assessed at the protein level in cell lysates by Western blotting using a custom rabbit polyclonal antiserum (Cocalico Biologicals, Reamstown, Pa.) raised against mesotrypsin peptide acetyl-TQAECKASYPGKITNS-NH₂ conjugated to KLH (EZ Biolab, Carmel, Ind.). For transwell invasion assays, cells were suspended in DMEM+0.1% BSA, mixed with BPTI-K15R/R17G as noted, and 1×10⁵ cells/well were used to seed 24-well 8.0 micron transwell inserts (BD Biosciences) coated with 50 μg Matrigel (BD Biosciences). Cells were allowed to invade for 24 hours at 37° C. and 5% CO₂ toward a chemoattractant in the lower chamber comprised of NIH/3T3-conditioned serum-free medium containing 50 μg/mL ascorbic acid and 50 ng/mL SDF-1β (R&D Systems) before methanol fixation, crystal violet staining, photographing of filters and counting of cells on the underside of the filter using Image Pro Plus 6.3 software (MediaCybernetics).

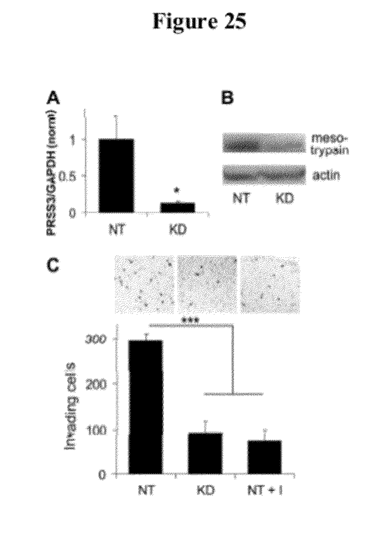

Using Capan-1 pancreatic cancer cells, which express high levels of PRSS3/mesotrypsin, the impact of PRSS3 knockdown by RNA interference and of mesotrypsin inhibition by BPTI-K15R/R17G treatment were measured on cellular invasion in Matrigel transwell invasion assays (FIG. 25). Transduction of Capan-1 cells with a lentiviral shRNA construct specifically targeting PRSS3 led to efficient suppression of PRSS3/mesotrypsin expression both at the transcript level (FIG. 25A) and at the protein level (FIG. 25B). In Matrigel transwell invasion assays, either PRSS3 knockdown or treatment with 100 nM BPTI-K15R/R17G led to equivalent inhibition of invasion by 70-75% relative to control cells (FIG. 25C). In aggregate, these results confirmed that BPTI-K15R/R17G inhibits malignant growth and invasion in cancer models in which PRSS3/mesotrypsin was been found to play a role in promoting these hallmarks of cancer.

### Example 11

### Selection of Mesotrypsin Inhibitors from a BPTI Phage Display Library Diversified at Residues 16 and 34

Residue 15 (P₁), Lys in BPTI-wt, was mutated to Arg in the library template. It was hypothesized that Lys or Arg would be accepted by mesotrypsin at this position, although Arg may be preferred. Subsite specificity and binding loop motifs were identified in the screen. All selected inhibitors possessed either Thr, Ser, Val, or Cys at residue 16 (the P₁′ position). Residue selection at this position (from among the four residues found) was not correlated with residue selection at any of the other diversified positions (i.e., either T, S, V, or C at P₁′) could be found in combination with any of the motifs described below. In addition, Pro (P) was also found at residue 16 (the P₁′ position), in combination with several of the listed motifs.

The following amino acid sequence motifs were identified by at least three independent selected phage DNA sequences when the library was screened under conditions designed to select for maximum affinity:

| Position: | 17 - | 18 - | 19 | - - - | 34 |
|---|---|---|---|---|---|
| Residue: | G - | R - | S | - - - | (E/T) |
| | G - | R - | A | - - - | E |
| | G - | N - | S | - - - | D |
| | G - | E - | T | - - - | (I/M) |
| | S - | Q - | V | - - - | L |
| | S - | N - | L | - - - | L |
| | S - | R - | G | - - - | (H/T) |
| | (S/C/G) - | R - | Y | - - - | (G/S/T) |
| | R - | L - | G | - - - | H |
| | R - | N - | A | - - - | H |
| | (K/R) - | R - | (G/Y/W) | - - - | Q |
| | A - | L - | T | - - - | K |

The following amino acid sequence motifs were identified by at least three independent selected phage DNA sequences when the library was screened under conditions designed to select for maximum proteolytic stability to mesotrypsin cleavage:

| Position: | 17 - | 18 - | 19 | - - - | 34 |
|---|---|---|---|---|---|
| Residue: | P - | (P/I) - | (G/I) | - - - | (L/V) |
| | P - | P - | S | - - - | (V/F/L) |
| | P - | I - | A | - - - | F |
| | P - | P - | C | - - - | G |

These results demonstrate that specific BPTI binding loop motifs are compatible with the goal of maximizing inhibitor affinity and proteolytic resistance to mesotrypsin. These motifs incorporated into BPTI can yield potent mesotrypsin inhibitors of potential therapeutic utility. Additionally, given the data reported herein showing that Kunitz domain binding loops and scaffolds can be independently optimized for maximal inhibitor affinity and stability to mesotrypsin, these identified binding loop motifs may yield potent mesotrypsin inhibitors of potential therapeutic utility when incorporated at corresponding positions of other Kunitz domains.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating cancer, wherein said method comprises administering a mesotrypsin inhibitor to said mammal, thereby treating said cancer.

2. The method of claim 1, wherein said mammal is a human.

3. The method of claim 1, wherein said cancer is prostate cancer or breast cancer.

4. The method of claim 1, wherein said inhibitor is BPTI-K15R/R17G.

5. A method for reducing cancer cell growth, wherein said method comprises administering a mesotrypsin inhibitor to a mammal having cancer cells under conditions wherein growth of said cancer cells within said mammal is reduced.

6. The method of claim 5, wherein said mammal is a human.

7. The method of claim 5, wherein said cancer cells are prostate cancer cells or breast cancer cells.

8. The method of claim 5, wherein said inhibitor is BPTI-K15R/R17G.

9. A method for reducing mesotrypsin activity, wherein said method comprises contacting mesotrypsin with a mesotrypsin inhibitor comprising a Kunitz domain under conditions wherein the activity of said mesotrypsin is reduced, wherein said mesotrypsin inhibitor has a Kᵢ less than 2×10⁻⁵ M and a k_{cat} (s⁻¹) value less than 5×10⁻³.

10. The method of claim 9, wherein said mesotrypsin inhibitor has a Kᵢ less than 5×10⁻⁷ M and a k_{cat} (s⁻¹) value less than 1×10⁻³.

11. The method of claim 9, wherein said mesotrypsin inhibitor is administered to a mammal to contact said mesotrypsin.

12. The method of claim 11, wherein said mammal is a human.

13. The method of claim 9, wherein said inhibitor is BPTI-K15R/R17G.

14. A method for identifying a human having had a prostatectomy as being likely to experience prostate cancer recurrence, wherein said method comprises:
(a) detecting the presence of an elevated level of PRSS3 nucleic acid expression in said patient, and
(b) classifying said human as being likely to experience prostate cancer recurrence based at least in part on said presence of said elevated level of PRSS3 nucleic acid expression.
